SEQUENCE LISTING (10 sequences)

SEQ ID NO: 1 - protein, 10 aa, Homo sapiens
ALKALPMHIR

SEQ ID NO: 2 - protein, 3 aa, Homo sapiens
ALK

SEQ ID NO: 3 - protein, 7 aa, Homo sapiens
ALPMHIR

SEQ ID NO: 4 - protein, 3 aa, Homo sapiens
HIR

SEQ ID NO: 5 - protein, 14 aa, Homo sapiens
VLVLDTDYKKYLLF

SEQ ID NO: 6 - protein, 4 aa, Homo sapiens
DTDY

SEQ ID NO: 7 - protein, 5 aa, Homo sapiens
DTDYK

SEQ ID NO: 8 - protein, 6 aa, Homo sapiens
GLDIQK

SEQ ID NO: 9 - protein, 4 aa, Homo sapiens
DIQK

SEQ ID NO: 10 - protein, 6 aa, Homo sapiens
IPAVFK

(19) United States
(12) Patent Application Publication
(10) Pub. No.: US 2009/0297545 A1
(43) Pub. Date: Dec. 3, 2009

(54) IMMUNOMODULATORY DAIRY PEPTIDES AND USES THEREOF

(75) Inventors: Sylvie Gauthier, Quebec (CA); Yvan Boutin, Quebec (CA); Arnaud Jacquot, Rennes (FR); Diane Saint-Sauveur, Paris (FR); Réjean Drouin, Quebec (CA); Ismail Fliss, Quebec (CA)

Correspondence Address: DAVID M. CARTER;CARTER & SCHNEDLER, P.A., P.O. BOX 2985, ASHEVILLE, NC, 28802, US

(73) Assignee: Universite Laval, Quebec (CA)

(21) Appl. No.: 12/472,868

(22) Filed: May 27, 2009

Related U.S. Application Data
(60) Provisional application No. 61/056,224, filed on May 27, 2008.

(51) Int. Cl. A61K 39/00; C07K 7/06; C07K 7/08; C07K 14/00; A61K 38/16; A61K 38/10; A61K 38/08; A61P 37/04; A61K 38/06; C07K 5/08; C07K 5/10

(52) U.S. Cl. 424/185.1; 530/328; 530/327; 530/326; 530/325; 530/324; 530/329; 514/16; 514/15; 514/14; 514/13; 514/12; 514/18; 530/330; 530/331

(57) ABSTRACT

The present invention provide a new method for modulating the immune system of a subject in need thereof by administering a dairy-derived peptide to the subject, particularly a β-lactoglubulin-derived peptide (β-Lg peptide). According to one aspect, the modulation of the immune system is the modulation of a Th1 response. According to another aspect, the modulation of the immune system is the modulation of a Th2 response.

### FIELD OF THE INVENTION

The present invention relates to peptides isolated from dairy products and having an immunomodulatory effect on a subject, such as a human subject. Uses of these immunomodulatory peptides, as well as methods for modulating the immune system, or the immune response of a subject such as a human subject to a stimulus, are provided.

### BACKGROUND

Bovine milk proteins are well-known to exert several physiological functions and to contain numerous bioactive peptides, which are encrypted within their primary sequence. Biological activities identified to date for milk peptides included antioxidative, antihypertensive, antimicrobial, opioid, mineral-carrying and immunomodulatory activities.

Both in vitro and in vivo studies have demonstrated that some whey protein-based ingredients, such as whey protein concentrate (WPC) and whey protein isolate (WPI), and individual whey proteins such as β-lactoglobulin (β-Lg), α-44 lactalbumin (α-La) and lactoferrin (Lf) can influence the immune response. Several studies have also shown that when these proteins are ingested, enzymatic hydrolysis in the gut releases short-chain peptide sequences with antimicrobial, opioid, antihypertensive, antithrombotic and immunomodulatory activities, suggesting the enzymatic release of bioactive peptides from these proteins (Gauthier et al., 2006). However, few studies have evaluated the effect of peptides present in the primary sequence of the major whey proteins on the modulation of the immune system.

Immunoregulatory peptides affecting both the innate and acquired immune response have been described in the sequences of whey proteins (Gauthier et al., 2006; Gill et al., 2000). However, only a limited number of studies have identified the actual sequence of the peptides responsible for the immunomodulatory effects. For instance two peptides isolated from α-lactalbumin Tyr-Gly (f50-51; f18-19) and Tyr-Gly-Gly (f18-20) were found to stimulate the proliferation of cultured human blood lymphocytes (Kayser & Meisel, 1996). Gly-Leu-Phe (f51-53) is another immunostimulating peptide derived from bovine α-lactalbumin: it binds to specific sites on human neutrophils and monocytes (Jaziri et al., 1992), protects mice against Klebsiella pneumonia infection (Berthou et al., 1987), stimulates superoxide anion production by neutrophils (Migliore-Samour et al., 1992), and human monocyte-macrophage adherence and phagocytosis of human senescent red blood cells (Gattegno et al., 1988).

Whey proteins and peptides can be promising candidates to manage infections, not only because of their antibacterial activity (Early et al., 2001; Kutila et al., 2003), but also for their ability to modulate the immune response (Gauthier et al., 2006). Both aspects can influence the resistance to an infection, and this bifunctionality was demonstrated by Biziulevi\check{c}ius et al. (2006). They showed that hydrolyzed whey proteins such as α-lactalbumin and β-lactoglobulin stimulated the autolytic system of all naturally autolyzing and some naturally non-autolyzing microbial strains. Furthermore, when these protein hydrolysates were given orally to mice, they increased the phagocytic ability of peritoneal macrophages. The authors noted a correlation between these antimicrobial and immunostimulatory activities.

However, the whey proteins effect on the immune response to an infection can not always be attributed to an enhanced antigen-specific antibody production. For example, in rotavirus-infected mice, oral supplementation with a WPC reduced the frequency of severe diarrheal symptoms, onset of severe symptoms, and duration of high viral shedding in the feces (Wolber et al., 2005). However, in this latter study, local and systemic anti-rotavirus antibody levels remained unaffected by the WPC, which led the authors to suggest that the WPC might have increased neutrophil or natural killer cell activity.

### BRIEF SUMMARY

It is an aspect of the invention to provide a peptide having the sequence as set forth in SEQ ID NO:2 and optionally comprising up to seven additional amino acids at the carboxy-terminal end. It is a further aspect of the invention to provide a peptide having the sequence as set forth in SEQ ID NO:4 and optionally comprising up to seven additional amino acids at the amino-terminal end. It is yet a further aspect of the invention to provide a peptide having the sequence as set forth in SEQ ID NO:3 and optionally comprising up to three additional amino acids at the amino-terminal end. It is still another aspect of the invention to provide a peptide having the sequence as set forth in SEQ ID NO:1.

It is another aspect of the invention to provide a peptide having the sequence as set forth in SEQ ID NO:6 and optionally comprising up to four additional amino acids at the amino-terminal end and/or up to six additional amino acids at the carboxy-terminal end. It is a further aspect of the invention to provide a peptide having the sequence as set forth in SEQ ID NO:7 and optionally comprising up to four additional amino acids at the amino-terminal end and/or up to five additional amino acids at the carboxy-terminal end. It is yet a further aspect of the invention to provide a peptide having the sequence as set forth in SEQ ID NO:5.

It is an aspect of the invention to provide a peptide having the sequence as set forth in SEQ ID NO:9 and optionally comprising up to two additional amino acids at the amino-terminal end. It is a further aspect of the invention to provide a peptide having the sequence as set forth in SEQ ID NO:8.

It is another aspect of the invention to provide a composition comprising a peptide having a sequence as set forth in any of SEQ ID NO:1 to SEQ ID NO:9, or salts thereof.

It is another aspect of the invention to provide a composition consisting of a peptide selected from the group consisting of: any of SEQ ID NO:1 to SEQ ID NO:9, and a salt thereof.

It is another aspect of the invention to provide a composition comprising a peptide having the sequence as set forth in SEQ ID NO:2 and optionally comprising up to seven additional amino acids at the carboxy-terminal end. It is a further aspect of the invention to provide a composition comprising a peptide having the sequence as set forth in SEQ ID NO:4 and optionally comprising up to seven additional amino acids at the amino-terminal end. It is yet a further aspect of the invention to provide a composition comprising a peptide having the sequence as set forth in SEQ ID NO:3 and optionally comprising up to three additional amino acids at the amino-terminal end.

It is another aspect of the invention to provide a composition comprising a peptide having the sequence as set forth in SEQ ID NO:6 and optionally comprising up to four additional amino acids at the amino-terminal end and/or up to six additional amino acids at the carboxy-terminal end. It is a further aspect of the invention to provide a composition comprising a peptide having the sequence as set forth in SEQ ID NO:7 and optionally comprising up to four additional amino acids at the amino-terminal end and/or up to five additional amino acids at the carboxy-terminal end.

It is another aspect of the invention to provide a composition comprising a peptide having the sequence as set forth in SEQ ID NO:9 and optionally comprising up to two additional amino acid at the amino-terminal end.

It is another aspect of the invention to provide an adjuvant composition comprising a peptide as defined herein, or salts thereof. Such an adjuvant composition can be used in conjunction with a vaccine, or as a part of a vaccine, in order to increase or stimulate the immune response induced by the vaccine.

It is another aspect of the invention to provide a composition for inducing, increasing or stimulating the secretion of Th1-cytokines, such as interleukin-2 (IL-2), interferon-γ (INF-γ), tumor necrosis factor α (TNF-α) and granulocyte-macrophage colony-stimulating factor (GM-CSF) for example, and comprising/consisting essentially of/consisting of a peptide as defined herein, or salts thereof. The secretion of Th1-cytokines is preferably performed by immune cells, such as, for example, T lymphocytes.

It is another aspect of the invention to provide a method for inducing, increasing or stimulating a Th1 response in a subject in need thereof by administering an immunostimulatory-effective amount of a composition comprising/consisting essentially of/consisting of a peptide as defined herein, or salts thereof to the subject. The immunostimulatory-effective amount of composition to be administered is sufficient to produce the desired effect, i.e. the inducing, increasing or stimulating of the Th1 response in the subject, and should be understood as such. The necessary amount is therefore easily identifiable by those of skill in the art, and can be, as a non-limitative example, of about between 0.005 to 5 g per day of purified peptides or of peptidic fractions enriched in immunomodulatory peptides.

It is another aspect of the invention to provide a method for treating, alleviating or preventing an infection in a subject in need thereof by administering an immunostimulatory-effective amount of a composition comprising/consisting essentially of/consisting of a peptide as defined herein, or salts thereof to the subject. For example, and in a non-limitative manner, the infection can be caused by a microorganism selected from the group consisting of Escherichia coli, such as Escherichia coli 0157:H7, Streptococcus pneumoniae, Clostridium difficile or influenza virus. The immunostimulatory-effective amount of composition to be administered is sufficient to produce the desired effect, i.e. the treatment, alleviation or prevention of the infection in the subject, and should be understood as such. The necessary amount is therefore easily identifiable by those of skill in the art, and can be, as a non-limitative example, of about between 0.005 to 5 g per day of purified peptides or of peptidic fractions enriched in immunomodulatory peptides.

It is another aspect of the invention to provide a method for treating, alleviating or preventing an allergy in a subject in need thereof by administering an immunostimulatory-effective amount of a composition comprising/consisting essentially of/consisting of a peptide as defined herein, or salts thereof to the subject. For example, the allergy can be chronic allergy or acute allergy, an allergy to a food or aliment (e.g. peanut), to pollen (e.g. birch), to dust mites or to animal-derived allergens (e.g. cat). The immunostimulatory-effective amount of composition to be administered is sufficient to produce the desired effect, i.e. the treatment, alleviation or prevention of the allergy in the subject, and should be understood as such. The necessary amount is therefore easily identifiable by those of skill in the art, and can be, as a non-limitative example, of about between 0.005 to 5 g per day of purified peptides or of peptidic fractions enriched in immunomodulatory peptides.

In administering a composition according to any aspect of the invention, it will be apparent to the skilled reader that the administration can be any type of administration, in conjunction or not with any pharmaceutically and biologically acceptable carrier. Preferably, the administration is oral administration, nasal administration, enteral administration, rectal administration, vaginal administration and transmucosal administration. Also preferably, the subject according to any aspect of the invention is preferably a human subject.

It is another aspect of the invention to provide a composition for inducing, increasing or stimulating the secretion of Th2-cytokines, such as interleukin-4 (IL-4), interleukin-5 (IL-5), interleukin-6 (IL-6), interleukin-10 (IL-10), interleukin-13 (IL-13) and transforming growth factor β (TGF-β), and comprising/consisting essentially of/consisting of a peptide as defined herein, or salts thereof. The secretion of Th2-cytokines is preferably performed by immune cells, such as, for example, T lymphocytes.

It is another aspect of the invention to provide a method for inducing, increasing or stimulating a Th2 response in a subject in need thereof by administering an immunostimulatory-effective amount of a composition comprising/consisting essentially of/consisting of a peptide as defined herein, or salts thereof to the subject. The immunostimulatory-effective amount of composition to be administered is sufficient to produce the desired effect, i.e. the inducing, increasing or stimulating of the Th2 response in the subject, and should be understood as such. The necessary amount is therefore easily identifiable by those of skill in the art, and can be, as a non-limitative example, of about between 0.005 to 5 g per day of purified peptides or of peptidic fractions enriched in immunomodulatory peptides.

It is another aspect of the invention to provide a method for inducing, increasing or stimulating the production of immunoglobulin A in a subject in need thereof by administering an immunostimulatory-effective amount of a composition comprising/consisting essentially of/consisting of a peptide as defined herein, or salts thereof to the subject. The immunostimulatory effectiveness is preferably a simple, effective and non-toxic increase of the immune response of the subject, particularly following mucosal administration of the composition, without having to co-administrate another molecule to potentially mask or alter a desirable epitope. The immunostimulatory-effective amount of composition to be administered is sufficient to produce the desired effect, i.e. the inducing, increasing or stimulating of the production of immunoglobulin A in the subject, and should be understood as such. The necessary amount is therefore easily identifiable by those of skill in the art, and can be, as a non-limitative example, of about between 0.005 to 5 g per day of purified peptides or of peptidic fractions enriched in immunomodulatory peptides.

It is another aspect of the invention to provide a method for treating, alleviating or preventing an inflammatory disease in a subject in need thereof by administering an immunostimulatory-effective amount of a composition comprising/consisting essentially of/consisting of a peptide as defined herein, or salts thereof to the subject. For example, the inflammatory disease can be a VEGF-associated inflammatory disease. As further non-limitative examples, the inflammatory disease can be atherosclerosis, arthritis, eczema and inflammatory bowel disease. The immunostimulatory-effective amount of composition to be administered is sufficient to produce the desired effect, i.e. the treatment, alleviation or prevention of the inflammatory disease in the subject, and should be understood as such. The necessary amount is therefore easily identifiable by those of skill in the art, and can be, as a non-limitative example, of about between 0.005 to 5 g per day of purified peptides or of peptidic fractions enriched in immunomodulatory peptides.

It is another aspect of the invention to provide a method for inducing, increasing or stimulating the proliferation of immune cells, such as splenocytes for example in a subject in need thereof, said method comprising the step of administering an immunostimulatory-effective amount of a composition comprising/consisting essentially of/consisting of a peptide as defined herein, or salts thereof to the subject. The immunostimulatory-effective amount of composition to be administered is sufficient to produce the desired effect, i.e. the inducing, increasing or stimulating of the proliferation of immune cells in the subject, and should be understood as such. The necessary amount is therefore easily identifiable by those of skill in the art, and can be, as a non-limitative example, of about between 0.005 to 5 g per day of purified peptides or of peptidic fractions enriched in immunomodulatory peptides.

### BRIEF DESCRIPTION OF THE DRAWINGS

Having thus generally described the nature of the invention, reference will now be made to the accompanying drawings, showing by way of illustration, a particular embodiment thereof, and in which:

FIG. 1 illustrates the stimulation indices (SI) measured for the peptide β-Lg f78-83 (SEQ ID NO. 10; control peptide) at different concentrations (10-2000 μg/mL) on the proliferation of (A) resting- and (B) ConA (0.5 μg/mL)-stimulated splenocytes, validating the use of this peptide as a control peptide for further experiments. Data are expressed as means±SD (n≧4).

FIG. 2 illustrates the absence of effect of the control peptide (β-Lg f78-83; SEQ ID NO. 10) on murine splenocytes: A) Cells after 96 h of incubation without ConA (4×); B) Cells after 96 h of incubation without ConA in the presence of 2000 μg/mL of peptide β-Lg f78-83; SEQ ID NO. 10(4×); C) Cells after 72 h of incubation in the presence of 0.5 μg/mL of ConA (4×); D) Cells after 72 h of incubation in the presence of 0.5 μg/mL of ConA and 2000 μg/mL of peptide β-Lg f78-83; SEQ ID NO. 10 (4×).

FIG. 3 illustrates a schematic representation of the protocol in non-infected group and the group infected with E. coli O157:H7.

FIG. 4 illustrates a post-infection weight variations in E. coli-infected groups. Significant difference (* p<0.05; ** p<0.01) between the samples and their corresponding PBS control are indicated.

FIG. 5 illustrates post-infection food intake in E. coli-infected groups.

FIG. 6 illustrates serum IgA titers in non-infected (A) and E. coli-infected (B) groups. Significant difference (* p<0.05; ** p<0.01) between the samples and their corresponding PBS control are indicated.

FIG. 7 illustrates chromatograms of the whey protein hydrolysate (A), the fractions F1 (pI<4.5) (B), F2 (4.5<pI<7) (C) and F3 (pI>7) (D).

FIG. 8 illustrates the effects of different concentrations (10-2000 μg/mL) of the β-lactoglobulin peptides on the proliferation of resting murine splenocytes. Stimulation indices (SI) are expressed as means+SEM (n=4). Significant differences (* p<0.05 and ** p<0.01) between the samples and the control cells are indicated.

FIG. 9 illustrates the effects of different concentrations (10-2000 μg/mL) of the β-lactoglobulin peptides on the proliferation of concanavalin A-stimulated (0.5 μg/mL) splenocytes. Stimulation indices (SI) are expressed as means+SEM (n=4). * Significant difference (* p<0.05 and ** p<0.01, respectively) between the samples and the control cells+ConA are indicated.

FIG. 10 illustrates the known sequence of β-lactoglobulin.

### DETAILED DESCRIPTION

In accordance with the present invention, there is provided a new method for modulating the immune system of a subject in need thereof by administering a dairy-derived peptide to the subject, particularly a β-lactoglubulin-derived peptide (β-Lg peptide). According to one aspect, the modulation of the immune system is the modulation of a Th1 response. According to another aspect, the modulation of the immune system is the modulation of a Th2 response.

A Th1 response, also referred to as a cellular response, is an immune response that traditionally does not involve antibodies, but rather involves the activation of macrophages, natural killer cells, antigen-specific cytotoxic T-lymphocytes, and the release of various cytokines in response to an antigen. Non-limitative examples of cytokines associated with a Th1-response include interferon-γ (INF-γ), tumor necrosis factor (TNF) and interleukin-2 (IL-2).

A Th2 response, also referred to as a humoral response, is an immune response that traditionally involve secreted antibodies, or immunoglobulins, produced by B cells. Non-limitative examples of cytokines associated with a Th2-response include IL-4, IL-5, IL-6, IL-10 and IL-13. Immunoglobulins are glycoproteins in the immunoglobulin superfamily that function as antibodies. They are found in the blood and tissue fluids, as well as many secretions. In mammals there are five types of antibody: IgA, IgD, IgE, IgG, and IgM. Each immunoglobulin class differs in its biological properties and has evolved to deal with different antigens. For example, IgA is playing a critical role in mucosal immunity. More IgA is produced than all other types of antibody combined. However, most sources still include IgG as the most common form of immunoglobulin. In its secretory form, IgA is the main immunoglobulin found in mucous secretions, including tears, saliva, colostrum, intestinal juice, vaginal fluid and secretions from the prostate and respiratory epithelium. It is also found in small amounts in blood. Because it is resistant to degradation by enzymes, secretory IgA can survive in harsh environments such as the digestive and respiratory tracts, to provide protection against microbes that multiply in body secretions.

According to one aspect, the enzymatic hydrolysis of β-Lg by the main pancreatic enzymes (trypsin, chymotrypsin, or a mixture of both) allows for the release of specific peptides having a modulatory effect on the immune system, particularly regarding the proliferation of splenocytes in mouse and the secretion of specific Th1- and Th2-cytokines in cell culture supernatants. Mass spectrometric analysis of an enzymatic hydrolyzate (trypsin/chymotrypsin) prepared from whey proteins revealed the peptide sequences having such immunomodulatory effects. Moreover, by using synthetic peptides, we identified two specific regions of β-Lg as comprising these immunomodulatory peptides. These regions have been identified as corresponding to amino acid regions 92-105 (SEQ ID NO. 5) and 139-148 (SEQ ID NO. 1) of β-Lg.

Accordingly, one aspect provides that peptides 92-105 (SEQ ID NO. 5), 139-148 (SEQ ID NO. 1) and 142-148 (SEQ ID NO. 3) significantly increased mice splenocytes proliferation, both in presence and absence of the ConA mitogen. Another aspect provides that peptide sequence 139-148 (SEQ ID NO. 1) significantly increased the secretion of IFN-γ in splenocytes cell culture supernatants, with and without the ConA mitogen. Another aspect provides that peptide sequence 146-148 (SEQ ID NO. 4), included in both sequences 139-148 (SEQ ID NO. 1) and 142-148 (SEQ ID NO. 3), increased mice splenocytes proliferation significantly in absence of ConA, but non-significantly in presence of ConA. Another aspect provides that peptide sequence 146-148 (SEQ ID NO. 4) was also shown to strongly stimulate IFN-γ secretion (Th1) while inhibiting IL-4 secretion (Th2) by splenocytes in culture in presence of the mitogen ConA. Another aspect provides that peptide sequence 139-141 (SEQ ID NO. 2), also included in sequence 139-148 (SEQ ID NO. 1), significantly increased the proliferation of mice splenocytes in absence of ConA, but not in presence of ConA, and does not have any effect on the secretion of Th1- or Th2-cytokines by splenocytes, by opposition with peptide sequences 139-148 (SEQ ID NO. 1) and 146-148 (SEQ ID NO. 4) which both significantly increased the secretion of Th1-cytokines. Another aspect provides that peptide sequence 96-99 (SEQ ID NO. 6), included in sequence 92-105 (SEQ ID NO. 5), increased mice splenocytes proliferation significantly in absence of ConA, but non-significantly in presence of ConA, just like peptide sequence 146-148 (SEQ ID NO. 4) did. Another aspect provides that peptide sequence 96-99 (SEQ ID NO. 6) significantly increased IL-4 (Th2) and IFN-γ (Th1) secretion.

Another aspect provides that peptide sequence 139-148 (SEQ ID NO. 1), and particularly the sequence 146-148 (His-Ile-Arg) (SEQ ID NO. 4) possess the capacity to increase a specific immune response by increasing the secretion of Th1-cytokines.

Another aspect provides that peptide sequence 92-105 (SEQ ID NO. 5), and particularly the sequence 96-99 (Asp-Thr-Asp-Tyr) (SEQ ID NO. 6) possess the capacity to increase a specific immune response by increasing the secretion of Th2-cytokines.

Additional aspects include additional peptide sequences having an immunomodulatory effect, as described in the hereinafter examples. For example, β-Lg peptide sequences 9-14 (SEQ ID NO. 8), 11-14 (SEQ ID NO. 9) and 96-100 (SEQ ID NO. 7) have also been demonstrated to have significant immunomodulatory effects.

It is an aspect to use the peptide sequences identified herein as increasing the secretion of Th1-cytokines in the treatment and prevention of infections or allergies, or in the manufacture of natural products or food products for the prevention of infections or allergies.

It is an aspect to use the peptide sequences identified herein as increasing the secretion of Th2-cytokines in the treatment and prevention of inflammatory diseases, or in the manufacture of natural products or food products for the prevention of inflammatory diseases.

It is an aspect to use the peptide sequences identified herein as adjuvants for increasing the immune system or the immune response to a pathogen agent, or in in the manufacture of compositions or vaccines for doing same.

The present invention will be more readily understood by referring to the following examples which are given to illustrate the invention rather than to limit its scope.

### Example I

### Immunomodulating Effects of Peptides from β-Lactoglobulin and α-Lactalbumin on Murine Splenocytes

The present study focused at investigating the effect of some peptides from β-Lg and α-La on proliferation of resting- and ConA-stimulated murine splenocytes. The peptides were chemically synthesized and selected according to their physicochemical characteristics (amino acid composition, molecular weight, hydrophobicity and charge) and their theoretical release from native whey proteins by trypsin and/or chymotrypsin hydrolysis in order to simulate the intestinal digestion. The peptides showing different cell stimulating profiles were also selected to evaluate their effects on the secretion of interleukin (IL)-2, IL-4, IL-10 and interferon-gamma (IFN-γ).

Peptides. Peptides were chemically synthesized by the Eastern Quebec Peptide Synthesis Facility (Sainte-Foy, QC, Canada) using a peptide synthesizer Applied Biosystems (Model 433A, Foster City, Calif., USA). All the peptides had a degree of purity higher than 80%.

Ex vivo murine splenocytes proliferation assay. The splenocytes were isolated from 6- to 12-weeks-old female BALB/c mice (Charles River, St-Constant, QC, Canada) and cultured in the absence and presence of suboptimal concentration of ConA (0.5 μg/mL). The peptides were solubilized in complete RPMI medium, agitated overnight at 4° C. then the solutions were adjusted to pH 7. Thereafter, the peptide solutions were added at various final concentrations (10-2000 μg/mL, protein basis) to the wells of 96-well round-bottomed microplates, which were incubated at 37° C. at 80% relative humidity in a 5% CO₂ atmosphere for 72 h (with ConA) or 96 h (without ConA). Cell proliferation was evaluated by the reduction of the fluorochrome Alamar Blue™ (BioSource international, Camarillo, Calif., USA), which was added into the wells for the last 24 h of the incubation periods. The fluorescence of the supernatants was monitored at an excitation wavelength of 560 nm and an emission wavelength of 590 nm using a fluorometer (Fluoroscan Ascent, Thermo Electron Inc., Milford, Mass., USA). The assays were performed in triplicate (three wells per sample) and data are expressed as stimulation indices (SI), and calculated using the following equations:

$$\begin{matrix}SI ( without ConA ) = \frac{{Fluorescence}_{cells + sample} - {Fluorescence}_{medium}}{{Fluorescence}_{cells} - {Fluorescence}_{medium}} & [ 1 ] \\ SI ( with ConA ) = \frac{{Fluorescence}_{cells + sample + ConA} - {Fluorescence}_{medium + ConA}}{{Fluorescence}_{cells} - {Fluorescence}_{medium}} & [ 2 ]\end{matrix}$$

Cytokine analysis. The supernatants from 72 h-splenocytes cultures were collected and their cytokine content was measured. The optimal incubation times were determined from preliminary assays with peptide fractions. The levels of IL-4, IL-10 and IFN-γ were determined using commercial ELISA kits (BioLegend, San Diego, Calif., USA) according to the manufacturer's instructions. Interleukin-2 levels were also measured by ELISA using a commercial kit (BioSource International, Camarillo, Calif., USA) according to the manufacturer's instructions. The results are the means of four or more assays (three wells per sample). The detection limits for IL-2, IL-4, IL-10 and IFN-γ were <8.0, <1.0, <30.0 and <4.0 pg/mL, respectively.

Statistical analysis. Differences between samples and their respective controls in the spleen cell proliferation (Tables 2 and 3) and cytokine assays (Table 5) were determined by one-way analysis of variance (ANOVA) and using the Dunnett's t test with the significance set at p<0.05 or p<0.01.

### Effects of Peptides on Ex Vivo Splenocytes Proliferation.

Eight peptides from β-Lg and α-La were selected according to their physicochemical characteristics (amino acid composition, molecular weight, net charge at pH 7 and hydrophobicity) and their theoretical release from native proteins by trypsin and/or chymotrypsin hydrolysis to simulate the intestinal digestion (Table 1). The selected peptides have molecular weight (MW) ranging from 674 to 1730 Da, are neutral, negatively or positively charged at pH 7.0, have an isoelectric point varying from 4.4 to 11.0 and different hydrophobicity values (0.95 to 2.03).

TABLE 1
Physicochemical characteristics of the peptides studied.

| | | MW^{a} | Net charge | Isoelectric | H_{φave}^{b} |
|---|---|---|---|---|---|
| Peptide | Amino acid sequence | (Da) | at pH 7 | point^{a} | (Kcal per residue) |
| β-Lg f78-83 | (K)^{c}IPAVFK | 673.85 | +1 | 8.75 | 2.03 |
| β-Lg f15-20 | (K)VAGTWY | 695.77 | 0 | 5.49 | 1.46 |
| β-Lg f55-60 | (L)EILLQK | 742.91 | 0 | 6.10 | 1.54 |
| β-Lg f84-91 | (K)IDALNENK | 916.00 | −1 | 4.37 | 0.95 |
| β-Lg f92-105 | (K)VLVLDTDYKKYLLF | 1730.08 | 0 | 5.93 | 1.77 |
| β-Lg f139-148 | (K)ALKALPMHIR | 1149.46 | +2 | 11.00 | 1.54 |
| β-Lg f142-148 | (K)ALPMHIR | 837.05 | +1 | 9.80 | 1.54 |
| α-La f10-16 | (F)RELKDLK | 901.07 | +1 | 8.59 | 1.22 |

^{a}Theoretical mass and isoelectric point were obtained from ExPASy Proteomics Server.
^{b}Average hydrophobicity was calculated according to the method of Bigelow (1967).
^{c}Amino acid before the peptidic sequence is shown in parentheses.

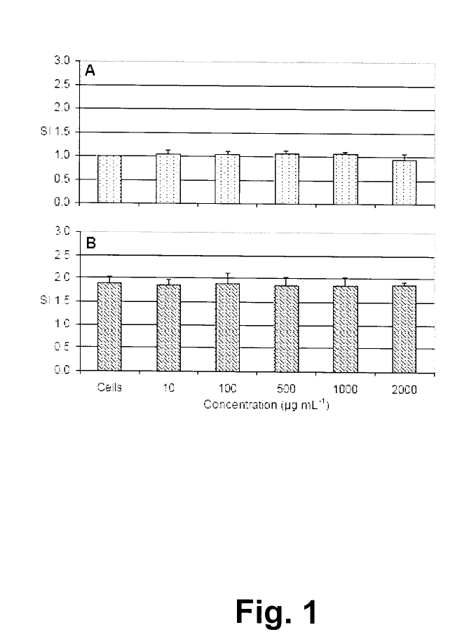

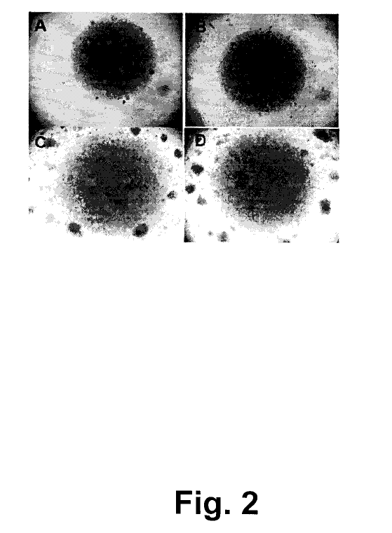

From the eight peptides under study, only the sequence β-Lg f78-83 (SEQ ID NO. 10) resulted in no effect on the proliferation of resting- and ConA-stimulated murine splenocytes at all the concentrations studied (FIGS. 1A and 1B). Microscopic observations of resting- and ConA-stimulated cells, with and without the presence of the highest concentration (2000 μg/mL) of the peptide β-Lg f78-83 (SEQ ID NO. 10), also showed that this peptide had no effect on the appearance of cultured cells (FIG. 2A to 2D). Consequently, this peptide was included in all the other proliferation assays at a concentration of 10 μg/mL and used as control for the statistical analysis.

The effects of the peptides on the proliferation of resting- and ConA-stimulated murine splenocytes, expressed as stimulation index (SI), are presented in Tables 2 and 3, respectively.

TABLE 2
Stimulation indices (means, n ≧ 4) measured for peptides from β-Lg
and α-La on the proliferation (96 h) of resting murine splenocytes.

| | | Peptide concentration (μg mL⁻¹) | | | | |
|---|---|---|---|---|---|---|
| Peptide | Control | 10 | 100 | 500 | 1000 | 2000 |
| β-Lg f15-20 | 1.02 | 1.01 | 1.12* | 1.46** | 1.65** | 1.42** |
| β-Lg f55-60 | 1.04 | 1.05 | 1.09 | 1.16 | 1.54** | 1.54** |
| β-Lg f84-91 | 1.00 | 0.99 | 1.08 | 1.20* | 1.22* | 1.27** |
| β-Lg f92-105 | 0.98 | 1.00 | 1.07 | 1.15** | 1.31** | 1.55** |
| β-Lg f139-148 | 0.98 | 1.08* | 1.17** | 1.35** | 1.53** | 1.77** |
| β-Lg f142-148 | 1.00 | 1.16 | 1.17 | 1.37** | 1.61** | 1.73** |
| α-La f10-16 | 1.11 | 1.06 | 1.19 | 1.47** | 1.81** | 1.97** |

^{a}Significant differences (*p < 0.05; **p < 0.01) between the samples and the control (peptide β-Lg f78-83 at 10 μg mL⁻¹).

TABLE 3
Stimulation indices (means, n ≧ 4) measured for peptides from
β-Lg and α-La on the proliferation (72 h) of murine splenocytes
in the presence of Concavanalin A (0.5 μg/mL).

| | | Peptide concentration (μg mL⁻¹) | | | | |
|---|---|---|---|---|---|---|
| Peptide | Control | 10 | 100 | 500 | 1000 | 2000 |
| β-Lg f15-20 | 1.68 | 1.65 | 1.71 | 1.67 | 1.78 | 1.77 |
| β-Lg f55-60 | 1.93 | 1.96 | 1.92 | 1.98 | 1.93 | 2.34** |
| β-Lg f84-91 | 2.10 | 2.20 | 2.27 | 2.24 | 2.18 | 2.16 |
| β-Lg f92-105 | 1.91 | 1.92 | 1.92 | 2.27** | 2.37** | 2.69** |
| β-Lg f139-148 | 2.10 | 2.28 | 2.24 | 2.36* | 2.62** | 2.73** |
| β-Lg f142-148 | 2.10 | 2.32 | 2.30 | 2.36 | 2.49** | 2.63** |
| α-La f10-16 | 1.93 | 1.85 | 1.95 | 1.95 | 2.08 | 2.26** |

^{a}Significant differences (*p < 0.05; **p < 0.01) between the samples and the control (peptide β-Lg f78-83 at 10 μg mL⁻¹ + ConA at 0.5 μg mL⁻¹).

The majority of the peptides stimulated at various degrees the proliferation of resting murine splenocytes (Table 2), generally with increasing concentrations (10-2000 μg/mL) of the peptides in the medium. The same tendencies were also observed for the proliferation of ConA-stimulated murine splenocytes (Table 3) but the results are generally only significant for the highest concentrations, indicating a lower stimulating effect of the peptides in the presence of ConA. The sequence β-Lg f139-148 (SEQ ID NO. 1) is representative of a group of other peptides (β-Lg f84-91, f92-105 (SEQ ID NO. 5), f142-148 (SEQ ID NO. 3) and α-La f10-16), which stimulated the proliferation of resting cells (Table 2) in a dose-dependent manner but at various degrees depending of the peptide. The most stimulating peptides of this group were the sequences β-Lg f139-148 (SEQ ID NO. 1), f142-148 (SEQ ID NO. 3) and α-La f10-16 for which the highest SI values were measured from 500 to 2000 μg/mL (Table 2). Among this group, the sequence β-Lg f139-148 (SEQ ID NO. 1) was the most potent peptide leading to significant difference in comparison with the control even at the lowest concentration studied (10 μg/mL). In ConA-stimulated cells (Table 3), the stimulating effect of the peptides β-Lg f139-148 (SEQ ID NO. 1) and f92-105 (SEQ ID NO. 5) remained highly significant for the highest concentrations studied (500-2000 μg/mL), while this effect decreased for the peptides β-Lg f142-148 (SEQ ID NO. 3) and α-La f10-16 and disappeared for the peptide β-Lg f84-91 (Table 3).

In order to establish some correlations between the physicochemical parameters of the peptides (Table 1) and their stimulating effect on murine splenocytes proliferation (Tables 2 and 3), linear and/or polynomial regression analyses were performed using the SI values obtained at the three optimal concentrations tested (500-2000 μg/mL) for all the peptides, except for the control peptide (β-Lg f78-83; SEQ ID NO. 10). For these correlations, the peptide concentrations were expressed either on protein basis or molarity basis to take into account the different molecular weight of each peptide. The most significant (>0.5) coefficients of determination (r2) obtained for all the conditions studied were presented in Table 4.

TABLE 4
Coefficients of determination (r2) calculated from the relationship between the
stimulation indices and some physicochemical parameters of the peptides studied^{a} at the
three optimal concentrations, which were expressed either as protein or molarity basis.

| | Peptide concentrations | | | | | | Peptide concentrations | | | | | |
|---|---|---|---|---|---|---|---|---|---|---|---|---|
| | (μg mL⁻¹, protein basis) | | | | | | (μg mL⁻¹, molarity basis^{b}) | | | | | |
| | Without ConA | | | With ConA | | | Without ConA | | | With ConA | | |
| Parameter | 500 | 1000 | 2000 | 500 | 1000 | 2000 | 500 | 1000 | 2000 | 500 | 1000 | 2000 |
| H_{φave} | 0.60P^{c} | 0.83P | 0.60P | —^{d} | — | — | 0.51P | 0.50P | 0.50P | 0.75P | 0.68P | 0.88P |
| Charge + | 0.60P | 0.51P | 0.52P | 0.80P | 0.59P | 0.80P | — | — | — | 0.51P | 0.50P | 0.50P |
| MW | — | — | — | — | — | — | 0.92L | 0.86L | 0.86L | 0.97L | 0.95L | 0.97L |
| AA^{e} | — | — | — | — | — | — | 0.90L | 0.80L | 0.81L | 0.98L | 0.97L | 0.96L |

^{a}Peptides included in the analyses of regression were α-La f10-16, β-Lg f15-20, β-Lg f55-60, β-Lg f84-91, β-Lg f92-105, β-Lg f139-148 and β-Lg f142-148. Physicochemical parameters used for these analyses are those reported in Table 1.
^{b}For each peptide, the concentrations in μg mL⁻¹ were calculated in molarity then the ratio SI/molarity was used for the analyses of regression.
^{c}Type of analysis of regression; P. polynomial; L. linear.
^{d}Represented r² value lower than 0.5.
^{e}Total number of amino acids.

Hydrophobicity and positive charge of the peptides seemed to be related to their stimulating effect on murine splenocytes. The most significant correlations were of polynomial type for both parameters and the r2 values varied from 0.50 to 0.88. Hydrophobicity seems to better correlate with peptide concentrations expressed on molarity basis since r2 values higher than 0.5 were obtained both for resting- and ConA-stimulated cells at the three optimal peptide concentrations studied. This result suggested that hydrophobicity is also related to the length of the peptides. For this parameter, the correlations indicated that a hydrophobicity value of about 1.8 lead the highest stimulating effect (result not shown). Conversely, the number of positive charge better correlated with peptide concentrations expressed on protein basis, suggesting that the length of the peptides is less important for this parameter. Nevertheless, for all the conditions studied, highest SI values were obtained with peptides bearing two to three positive charges (result not shown). The highest r2 values (0.80-0.98) were obtained for both the molecular weight and the total number of amino acids of the peptides, which are interrelated parameters. The correlations were of linear type and showed that higher is the length of the peptide and higher is their stimulating effect (result not shown). As expected, these correlations prevailed only by expressing peptide concentrations on a molarity basis. Overall, these results thus suggested that a long hydrophobic peptide bearing 2-3 positive charges has a higher potential to stimulate the proliferation of murine splenocytes and the immune system. However, these correlations give no information on the impact of the position of hydrophobicity and/or positive charge on the peptides that is probably also an important parameter to consider for their effect on the cell growth.

### Effects of Peptides on Ex Vivo Cytokine Secretion.

The peptides sequence β-Lg f139-148 (SEQ ID NO. 1) that previously showed stimulating effect on cell proliferation was selected to evaluate its effect on the ex vivo release of Th1 (IL-2 and IFN-γ) and Th2 (IL-4 and IL-10) cytokines. Cytokine analysis were performed at concentration leading to optimal spleen cells proliferation of peptide β-Lg f139-148 (SEQ ID NO. 1) (2000 μg/mL) as reported in Tables 2 and 3. The sequence β-Lg f78-83 (SEQ ID NO. 10), resulting in no effect on the cytokine secretion of resting- and ConA-stimulated murine splenocytes (results not shown), was used as control at concentration of 10 μg/mL for statistical analysis. The effects of the selected peptide on cytokine secretion from splenocytes cultured in the absence and presence of 0.5 μg/mL of ConA are shown in Table 5.

TABLE 5
Cytokine secretion measured in the supernatants of cultured
murine splenocytes by ELISA (means, n ≧ 4).

| | Splenocytes cultured | | Splenocytes cultured | |
|---|---|---|---|---|
| Cytokine | without ConA | | with ConA | |
| (pg mL⁻¹) | Control | β-Lg f139-148 | Control | β-Lg f139-148 |
| IL-4 | <1.0 | <1.0 | 4.4 | 3.0** |
| IL-10 | <30.0 | <30.0 | <30.0 | <30.0 |
| IL-2 | 10.0 | 9.3 | 15.6 | 15.8 |
| IFN-γ | 7.1 | 81.3** | 252.9 | 717.5** |

Cells were incubated for 72 h or 96 h without or with Concanavalin A (0.5 μg mL⁻¹) respectively, and in the Presence of the peptides β-Lg f139-148 (2000 μg mL⁻¹).

The sequence β-Lg f139-148 (SEQ ID NO. 1) strongly enhanced the release of IFN-γ in both resting- and ConA-stimulated splenocytes, whereas a slight inhibition of IL-4 secretion was observed in ConA-stimulated cells. The inhibitory effect of this peptide on IL-4 secretion could be interpreted with caution since the value is near the detection limit of this cytokine. Nevertheless, the effect on IFN-γ secretion of the peptide β-Lg f139-148 (SEQ ID NO. 1) may indicate a preferential effect on T-cells.

The present study highlights potential immunomodulatory effects of some new bioactive peptides encrypted in the primary sequence of the major two whey proteins, α-La and β-Lg. These peptides were demonstrated to stimulate the murine splenocyte proliferation and cytokine secretion at different degrees on both resting- and ConA-stimulated murine splenocytes. Splenocyte population was demonstrated to be mainly composed by lymphocytes (˜90%), in both resting- and ConA-stimulated spleen cells, with roughly 45% B cells and 44% T cells including T-helper and T-cytotoxic cells. As expected for a T-cell mitogen, they also demonstrated a very slight but non-significant increase in T-cell population in the presence of ConA, mainly for CD4+/CD25+ and CD8+/CD25+ probably because the use of very low mitogen concentration. The evaluation of the immune response modulation in our study thus especially targeted the cellular and humoral specific immune responses, which were mainly supported by T- and B-lymphocytes, respectively.

From the eight peptides under study only the peptide β-Lg f78-83 (SEQ ID NO. 10) showed no effect at any concentration, both on splenocyte proliferation and cytokine secretion. Seven peptides (β-Lg f15-20, f55-60, f84-91, f92-105, f139-148, f142-148, α-La f10-16) were found to stimulate cell proliferation at various degrees depending on the peptide concentration and the presence or absence of ConA.

The new biological activity of the peptides studied as cell proliferation promoters supports the view that many bioactive peptides are multifunctional. In fact, from the various peptides investigated in our study (Table 1), many of those have been already reported to have other biological activities. For example, the peptide β-Lg f15-20 was demonstrated to have antihypertensive (Pihlanto-Leppälä et al., 1998) and antimicrobial effects (Pelligrini et al., 2001), and the sequences β-Lg f142-148 (lactokinin) is well-known to exert antihypertensive activity (Mullally et al., 1997).

The mechanism by which the peptides identified stimulate cells proliferation is unknown but could be associated to host defence peptides (HDP). These peptides have a broad spectrum activity on the innate immunity against bacterial, fungal and viral pathogens that quickly eradicated pathogens through specific biochemical mechanisms. HDP are considered to have both direct antimicrobial activities (membrane and metabolic perturbations) and ability to direct prolonged cellular and humoral responses such as B-cell activation, antibody production, T-cytotoxic and natural-killer cells killing, and T-helper cell function. However, antimicrobial activity does not seem to be always related to their immunomodulating effect since the sequence β-Lg f78-83 was identified to have bactericidal activity by Pelligrini et al. (2001) while no effect on spleen cells proliferation and cytokine secretion was observed for this peptide in our study.

These results confirmed that several peptides encrypted in the primary sequence of α-La and β-Lg and theoretically released by digestive enzymes may influence the specific immune response through the modulation of splenocytes proliferation and cytokines secretion. They also supported the concept of multifunctional properties of food-derived peptides. It was also demonstrated that immunostimulating effect of peptides seems to be related to some physicochemical characteristics such as positive charge, hydrophobicity and the length of the peptides. Finally, this work highlighted the relevance of evaluating the effect of food-derived peptides on immune system by using purified cell phenotype or in vivo study in order to better understand their mechanism of action.

### Example 11

### Effect of Whey Peptidic Fractions Feeding on Immune Parameters in a Non-Infected and an Infected Murine Model

The first objective of the present study was to assess the immunomodulating effects of the whey protein isolate and the whey peptide fractions given orally to mice. The second goal was to evaluate the potential of these products on a murine model of enterohemorrhagic Escherichia coli O157:H7 infection.

Whey protein source and whey peptidic fractions preparation. Commercial microfiltered whey protein isolate (WPI) was kindly provided by Immunotech (Vaudeuil-Dorion, QC, Canada). The peptide fractions were prepared by hydrolyzing the WPI fraction using a mixture of trypsin:chymotrypsin (1:1), and stopping the reaction by ultrafiltration when the degree of hydrolysis reached 10%. The resulting hydrolysate was subsequently fractionated by liquid-phase isoelectric focusing a preparative Rotofor cell (Bio-Rad Laboratories, Hercules, Calif., USA). When voltage and current were stabilized, twenty peptide fractions were collected and their pH was measured immediately (e.g., before dropping due to atmospheric CO₂ absorption), then the fractions were pooled into three peptide fractions according to their pH: F1 (pH<4.5), F2 (4.5<pH<7), and F3 (pH>7).

Animals. Specific-pathogen-free, 6-week-old female BALB/c mice were obtained from Charles River (St-Constant, QC, Canada). All animal procedures were performed or supervised by qualified technicians following standard operation methods with respect to the Canadian Council on Animal Care and the Comité de protection des animaux de l'Université Laval. The experimental protocols were approved by our institutional animal care committee. The mice were housed individually in a temperature controlled environment (22±2° C.) with a 12 h light/dark cycle and fed ad libitum standard rodent chow (Charles River Laboratories, Wilmington, Mass., USA) with free access to water throughout the experiment.

Escherichia coli strain and growth conditions. E. coli O157:H7 (ATCC 35150) was obtained from the American Type Culture Collection (Rockville, Md., USA) and was maintained in 20% glycerol stock at −80° C. It was reactivated in Brain Heart Infusion (BHI, Difco Laboratories, Detroit, Mich., USA), and incubated aerobically at 37° C. The strain was subcultured three times, serially diluted (1:10), and plated onto sorbitol MacConkey agar. Agar plates were incubated overnight at 37° C., then a rose-red colony was picked and transferred into 10 mL BHI, incubated overnight at 37° C., and tested with the VIP EHEC Visual Immunoprecipitate Assay (BioControl Systems Inc., Bellevue, Wash., USA) performed according to the manufacturer's instructions. This assay confirmed the isolated colony was the Escherichia coli O157:H7 strain. The production of Shiga toxin proteins, both Stx1 and Stx2, was detected using the commercial Duopath Verotoxin Detection Kit (Merck, Darmstadt, Germany) according to the manufacturer's instructions. Briefly, 20 μL of the E. coli O157:H7 culture was inoculated in 1 mL of CAYE broth (20 g of Casamino acids, 6 g of yeast extract, 2.5 g of NaCl, 8.71 g of K₂HPO₄, and 1 mL of trace salts solution per liter of distilled water, which was composed of 5% MgSO₄, 0.5% MnCl₂, and 0.5% FeCl₃ dissolved in 0.0005 M H₂SO₄) and incubated for 6 h at 37° C. After incubation, 180 μL of the culture was mixed with 20 μL of polymyxin B solution and further incubated for 10 min at 37° C. A volume of 160 μL of the mixture was dispensed into the circular sample port on the test device. Results were observed after 20 min of incubation at room temperature.

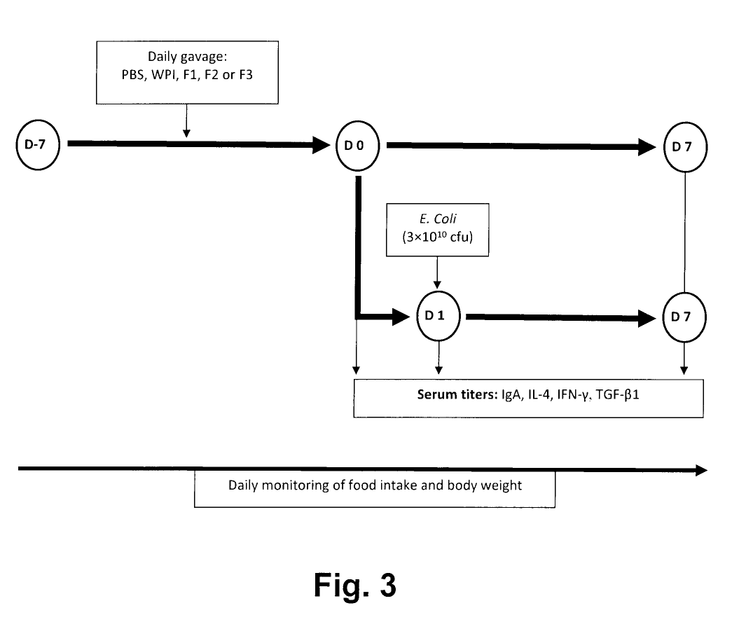

Mice feeding and infection procedure. After an acclimatization period of eight days, 130 mice were randomly assigned to one of the five following experimental groups corresponding to the different samples: phosphate-buffered saline (PBS, Sigma Chemical Co., St. Louis, Mo., USA), WPI, F1, F2 or F3. For the first seven days, mice were fed by gavage either sterile PBS or 3 mg of protein product (WPI, F1, F2 or F3) suspended in 0.3 mL of sterile PBS. At the end of the gavage period (day 0) and 7 days after the gavage had stopped (day 7), 7 mice in each group were sacrificed and blood was drawn for serum titer analyses (FIG. 3A).

In addition, the effect of the whey proteins and peptide fractions feeding prior to an infection was also assessed. On day 0, infection was induced with a single 0.3 mL dose of E. coli O157:H7 containing 3×10¹⁰ cfu suspended in sterile PBS given intragastrically. Six infected mice were then sacrificed on days 1 and 7 post-infection, and on these days blood was drawn for serum titer analyses (FIG. 3). The days of sacrifice were determined from preliminary assay showing that the symptoms of infection were the highest 24 h after the infection. Feed intake and animal body weight were monitored daily and expressed as average for each group throughout the experiment.

Immunoglobulin determination in the serum. On days 0, 1, and 7, mice were anaesthetized with IsoFlo isofluorane (Abbott Laboratories Ltd., St-Laurent, QC, Canada). Approximately 0.8 mL of blood was withdrawn via cardiac puncture then mice were subsequently sacrificed by cervical dislocation. Blood was centrifuged (1000×g for 10 min) and serum was stored at −20° C. Total IgA antibodies levels in response to the whey samples feeding and the E. coli O157:H7 infection were measured by Enzyme-Linked ImmunoSorbent Assay (ELISA) as described by LeBlanc et al. (2004). Briefly, IgA was quantified by sandwich ELISA using affinity-purified monoclonal goat anti-IgA (1.25 μg/mL) and horseradish peroxidase-conjugated anti-IgA-specific antibodies (1.25 μg/mL) as the primary and secondary antibodies, respectively. Serum samples were diluted 1:10,000, tested in triplicate, and IgA concentrations were extrapolated from a standard curve prepared using dilutions (0.98-62.5 ng/mL) of recombinant mouse kappa IgA. Mouse IgG was used as a negative control. All antibodies and standards were purchased from Sigma Chemical Co.

Cytokine production in the serum. Serum IL-4 levels were determined by ELISA using a commercial kit (BioLegend, San Diego, Calif., USA) according to the manufacturer's instructions. Serum IFN-γ was quantified by sandwich ELISA technique. Since murine TGF-β1 is 89.5% homologous to the human nucleotide sequence, TGF-β1 levels in the serum were measured by a sandwich ELISA using human TGF-β1 capture/detection antibody pairs (R&D Systems, Minneapolis, Minn., USA). For all the cytokines, absorbance was measured at 450 nm using a spectrophotometer plate reader (MultiSkan Spectrum model, Thermolabsystems, Brussels, Belgium). The detection limits for IL-4, IFN-γ and TGF-β1 were <0.1, <4 and <31.2 pg/mL, respectively.

Statistical analysis. Differences between samples and their respective PBS controls in the serum IgA and cytokine assays were determined by a one-way analysis of variance (ANOVA) using the least significant difference (LSD) test. In order to assess the evolution of the infection in time, the LSD test was also used to compare serum titers at different days for a given treatment.

### Effect of the Whey Protein Isolate and Peptide Fractions in Non-Infected Groups.

Body weight and food intake. During the first 7 days of gavage with the whey samples or PBS, mice body weight and food intake were stable, and remained stable for the 7 following days in all non-infected groups (data not shown).

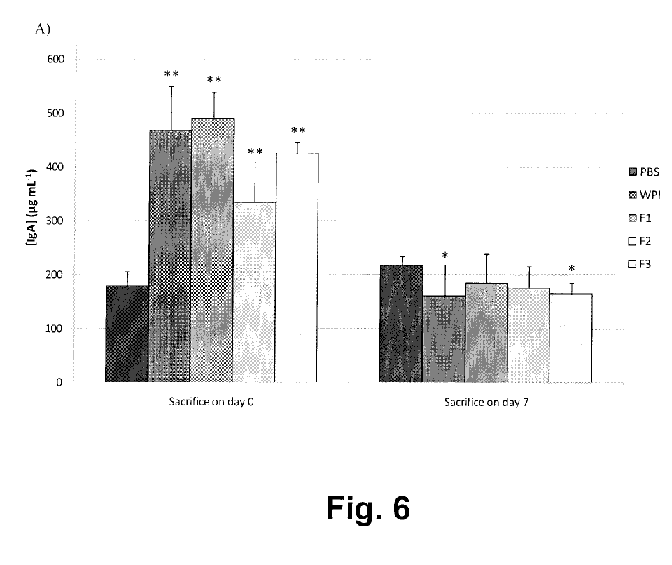

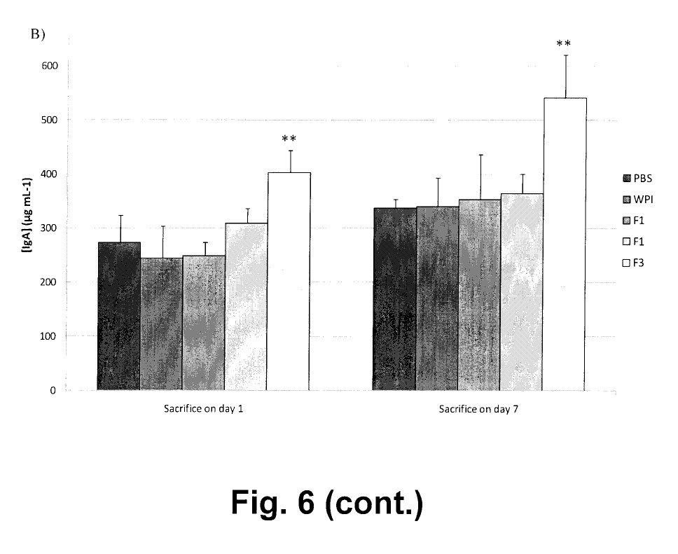

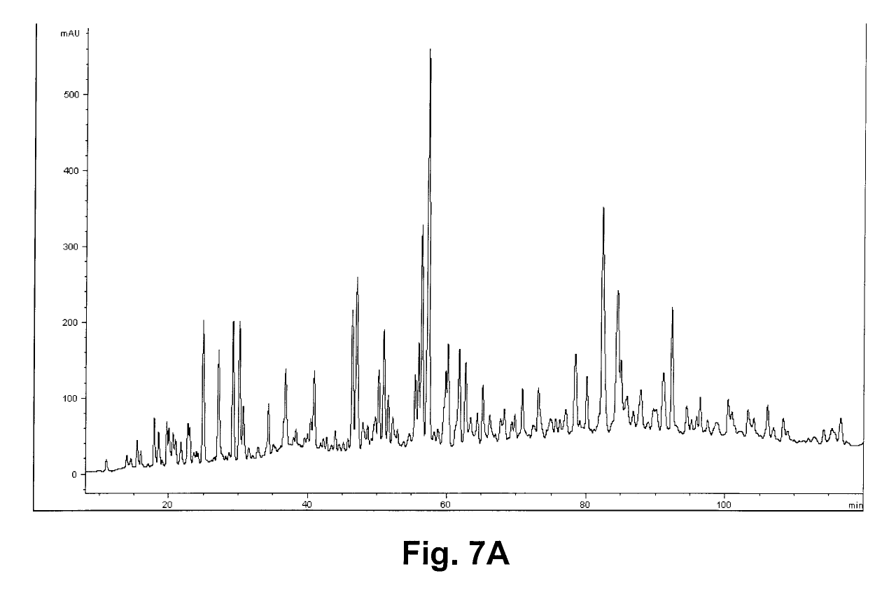

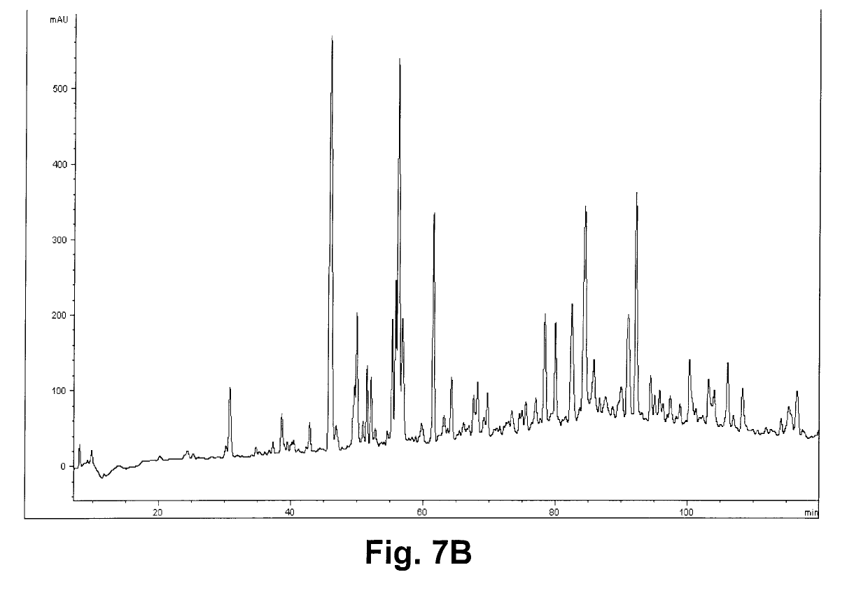

Effect of the whey protein isolate and peptide fractions on total serum IgA. In addition to the ad libitum standard rodent chow, the mice received the various products for 7 days prior to the infection (day 0) then returned to the standard rodent diet until the end of the experimentation (day 7). Total serum IgA levels were measured in non-infected groups on days 0 and 7 (FIG. 6A). On day 0, all products induced significantly higher IgA secretion (p<0.01), especially F1, WPI and F3 for which IgA production was more than doubled compared to the PBS control group. On day 7, serum IgAs were back to their original levels. This effect could be explained by the impact of individual whey proteins which have been reported to influence immunoglobulin secretion. It was previously shown that mice fed 4 mg of lactoferrin by daily intragastric gavage for 5 days exhibited higher levels of total IgA and IgG in the intestinal secretions as well as increased lactoferrin-specific antibody responses in the serum and intestinal secretions. Lactalbumin also has an impact on antibody production. Mice fed a diet high in lactalbumin pancreatic hydrolysate showed an increased specific humoral immune response to sheep red blood cells compared to mice fed a casein hydrolysate diet. The WPI used in the present study contained 0.6% lactoferrin and 26% α-lactalbumin, which could account for the impact of the whey products on IgA levels.

Our results not only confirm the capacity of whey proteins to enhance IgA secretion reported in the literature, they also suggest that this effect is maintained when proteins are digested with trypsin:chymotrypsin, and further separated into peptide fractions. Vinderola et al., (2006) demonstrated that oral administration of products derived from milk fermentation by kefir microflora (containing polysaccharide and milk peptidic fractions) to mice was able to modulate the gut mucosa immune response with an increase in the number of IgA⁺ cells in the small and large intestine lamina propria. The authors suggested that the induction of IgA⁺ B cells could enhance immune surveillance to prevent intestinal infections. However they could not determinate whether the immunomodulatory effects observed were attributable to the polysaccharide, the peptide fraction, or the combination of both. Duarte et al. (2006) observed that the oral administration of a fish powder concentrate was not able to modify the number of IgA⁺ cells in the small intestine lamina propria. Yet after the fish protein was fermented for 24 hours by yeast, the smaller oligopeptides that were released increased the number of IgA⁺ cells in the small intestine lamina propria of mice as well as the S-IgA content in the intestinal lumen. All these studies suggest that microbial cleavage of food proteins could promote the unfolding of these molecules and facilitate the release of bioactive peptides. Our results show that it is also the case for peptides released by a mixture of trypsin and chymotrypsin, since the three peptide fractions given orally to mice induced a significant (p<0.01) higher secretion of IgA.

Our results demonstrate the capacity of the WPI and the peptide fractions to induce higher total serum IgA levels in the absence of an infection. These products may therefore be used as adjuvants in order to enhance the immunogenicity of orally delivered vaccines. The B subunit of the cholera toxin is a classic example and has been extensively studied as a mucosal. The cholera toxin generates potent anti-toxin antibodies following systemic immunization, potentially providing a strategy to enhance immunogenicity of orally delivered antigens. One problem is the risk of inducing pathological diarrhea. However, this toxin acts as an adjuvant at doses lower than that required for the induction of toxic side effects, and the B subunit has been identified as the immune adjuvant agent, at least when administered intranasally or parenterally. In this study we observed that whey proteins and peptide fractions could be interesting alternatives for orally administered vaccines. Whey proteins have been shown to increase humoral immune responsiveness to T-dependent vaccine antigens such as influenza vaccine, diphtheria and tetanus toxoids, poliomyelitis vaccine, ovalbumin and cholera toxin sub-unit. Mice fed a WPC produced elevated levels of antigen-specific antibodies against all tested antigens in the intestinal tract and serum, compared to mice that were fed a standard chow diet.

Effect of the whey protein isolate and peptide fractions on serum cytokine production. Serum IFN-γ was measured in non-infected groups on days 0 and 7 (Table 6).

TABLE 6
Serum cytokine titers (ng/mL)

| | | Day of | | | | | |
|---|---|---|---|---|---|---|---|
| Cytokine | Group | analysis | PBS | WPI | F1 | F2 | F3 |
| IFN-γ | Not | 0 | 19.9 | 26.3 | 16.5 | 34.0*^{a} | 23.2 |
| | infected | 7 | 17.8 | 17.2 | 4.5* | 37.2** | 27.3 |
| | Infected | 1 | 27.8 | 31.9 | 19.0 | 17.0 | 10.0 |
| | | 7 | 1.1 | 6.2** | 0.8 | 0.7 | 0.7 |
| TGF-β1 | Not | 0 | 78.7 | 68.0 | 73.7 | 82.3 | 96.8** |
| | infected | 7 | 41.6 | 27.2** | 45.8 | 38.9 | 60.1** |
| | Infected | 1 | 57.2 | n.a.^{b} | 27.4 | 42.1 | 48.7 |
| | | 7 | 52.2 | 68.8 | 52.3 | 80.7 | 32.3 |

^{a}Significant difference (*p < 0.05; **p < 0.01) between the samples and their corresponding PBS control.
^{b}n.a.: not available.

In the absence of infection, the WPI did not affect IFN-γ levels. On the other hand, the peptide fractions issued from this product had contrasting effects: F1 significantly inhibited IFN-γ production on day 7, F2 significantly stimulated the secretion of IFN-γ in the serum on day 0 and more strongly on day 7, and F3 also increased the secretion of this cytokine on day 0 and 7 but the results were not significant compared to the PBS control. Hence the enzymatic digestion of the WPI with pancreatic enzymes releases peptides with both a stimulating and an inhibiting effect on the production of IFN-γ, which could account for the neutral effect of WPI in non-infected groups. Mice fed products derived from milk fermentation by kefir microflora (containing polysaccharide and milk peptidic fractions) for 7 days showed a significant increase of IL-4 and IFN-γ in the small intestine lamina propria of mice (Vinderola et al., 2006). This immunostimulation achieved in the gut mucosa was reflected in the serum as well, indicating that the effect of ingested peptides goes beyond the lumen of the intestine and extends to cytokines circulating in the serum. We did not detect IL-4 in the serum of any group (data not shown), infected or not, despite a detection limit of 0.1 pg/mL for the ELISA test, but we observed a stimulation of IFN-γ in the serum by the peptide fractions F2 and F3 in the absence of infection. In contrast with the effects of the whey samples on IgA production that were not visible 7 days after the end of the gavage, the impact on serum IFN-γ is still present and sometimes even stronger on day 7. These results are in agreement with previous findings which showed a strong IFN-γ stimulatory effect of the peptide fractions on in vitro murine splenocytes, especially with the F2 fraction.

Serum TGF-β1 was also measured in infected and non-infected groups (Table 6). WPI significantly inhibited TGF-β1 in non-infected mice on day 7, whereas F3 stimulated it on days 0 and 7. This has already been observed by Penttila et al. (2001) who demonstrated that a whey protein extract given orally may induce TGF-β1 secreting cells after oral sensitisation with an antigen, underlining TGF-β1's role in tolerance induction.

We have shown in this first part that oral administration of whey proteins and peptide fractions for 7 days in healthy mice have an impact on components of their immune system. The most striking result is the strong increase in total serum IgA in all mice that received the whey products, suggesting a state of enhanced immunological vigilance. The whey samples also affected key cytokines in the serum (IFN-γ and TGF-β1). Therefore the effects of the whey proteins and peptide fractions were assessed in a murine model of enterohemorrhagic Escherichia coli O157:H7 infection.

### Effect of the Whey Protein Isolate and Peptide Fractions in Infected Groups.

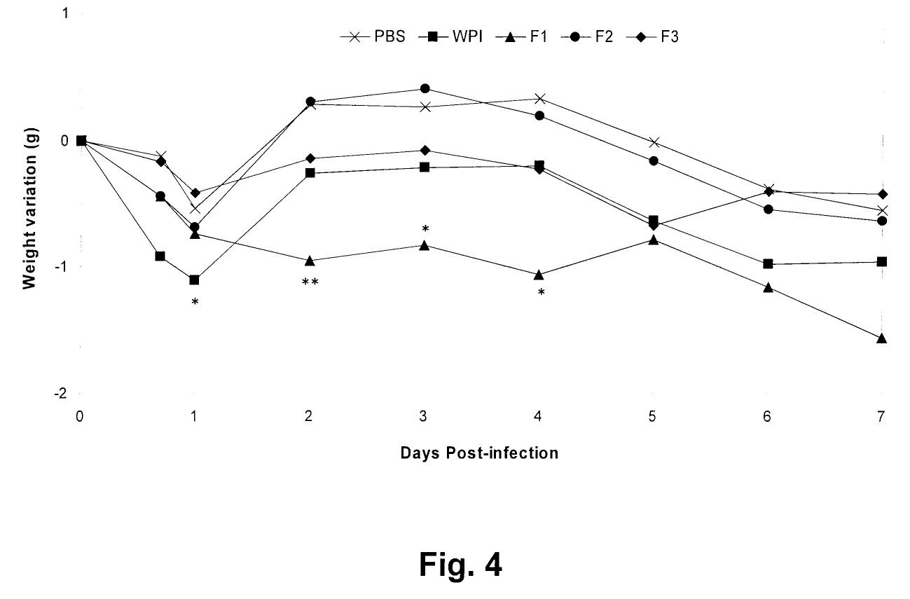

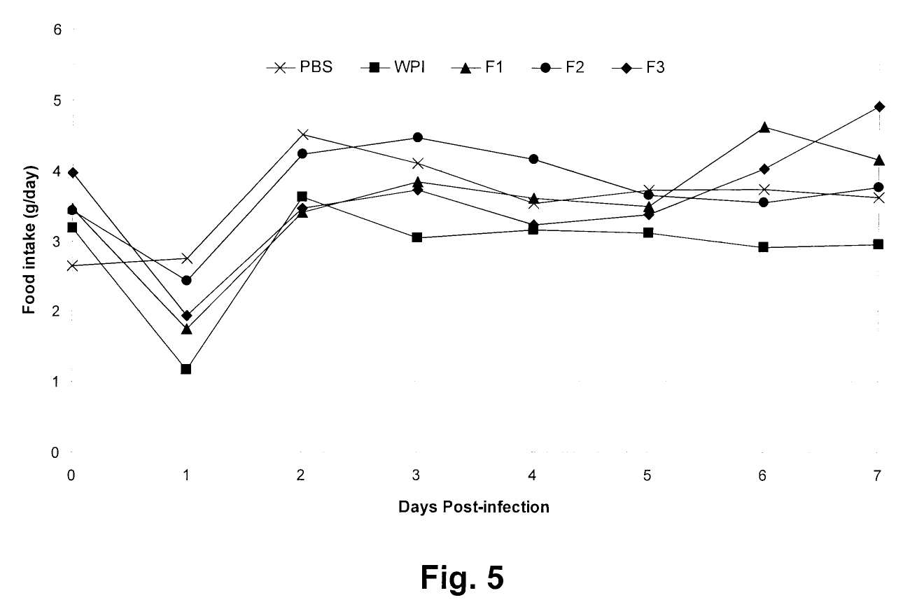

Body weight and food intake. As established in the preliminary assay, the highest weight loss was observed at 24 h post-infection (FIG. 4). This weight loss was particularly marked in the WPI group with a value significantly different (p<0.05) from the PBS control group. All infected groups went back to their pre-infection weight on day 2, except the group fed the peptide fraction F1 that kept losing weight up to 7 days after the infection with values significantly different from the PBS control group on days 2, 3 and 4. The reason for the sustained drop in body weight in the F1-fed group is unclear. It could be hypothesized that the peptides in the F1 fraction modified the intestinal cell lining, therefore allowing a better adhesion and/or translocation of the pathogen. Alternatively, the acidic nature of the F1 fraction (composed of peptides having a pI<4.5 obtained by isoelectrofocusing) might have disrupted the commensal intestinal microbiota, facilitating the subsequent growth and colonization of E. coli O157:H7. These observations highlight the importance of fractionating peptides after their enzymatic digestion, which may allow the isolation of potentially detrimental and beneficial peptides. The general tendency observed in body weight variations was reflected in food intake (FIG. 5), which dropped at 24 h then went back up to normal on day 2. However, no significant differences in food intake were noted between the WPI- or fraction-treated and the PBS control groups, and these results showed that the weight loss observed in the group fed the peptide fraction F1 (FIG. 4) can not be attributed to a decrease in their food intake.

In addition to the moderate weight loss and lower food intake at 24 h, some infection symptoms such as lethargy and ruffled fur were visible in all infected groups. One mouse died in each infected group (12 mice per group), and morbidity varied from 16 to 41% of infected mice (data not shown). These values are lower than those (˜70% on day 3) reported by Shu & Gill (2001) who used male mice challenged with lower dose (1×10⁹ cfu) of E. Coli O157:H7.

Taken together these results suggest that, despite a high microbial load given orally to the mice (3×10¹⁰ cfu), the resulting infection was not as severe as anticipated. Our study showed weight loss and a decrease in food intake, as well as morbidity and mortality in all infected groups. This indicates that mice were affected by the infection even though the symptoms were short-term and few significant differences were observed between the PBS control group and the groups fed the tested whey products. In order to further investigate the impact of these whey products on the systemic immune system of infected mice, IgA and some cytokines (IFN-γ, IL-4 and TGF-β1) were measured in the serum.

Effect of the whey protein isolate and peptide fractions on total serum IgA. Total serum IgA levels were measured in infected groups on days 1 and 7 (FIG. 6B). Infection induced a gradual increase in IgA serum levels in the control PBS group: 178 μg/mL on day 0, 273 μg/mL on day 1 and 337 μg/mL on day 7 post-challenge. This is not surprising since increased specific IgA antibodies are involved in the response to the infection by E. coli and even reduced recolonization after a secondary challenge. Serum IgA levels were higher on day 7 than on day 1 in all infected groups, indicating a delayed response even though the mice were back to their pre-infection body weights and did not show any symptoms of the infection anymore.

In the present study, even though the serum IgA levels were back to normal on day 7 in non infected groups, the infection represented a significant enough antigenic challenge to induce an enhanced IgA production even 7 days after the feeding of F3 had stopped. The basic peptide fraction F3 caused a significant increase in serum IgA in the infected mice compared to the PBS group on days 1 and 7. These results are consistent with the work of Leblanc et al. (2004), who studied the effects of 7-day administration of a peptidic fraction resulting from extensive proteolysis of milk by L. helveticus followed by a challenge with E. coli O157:H7. The peptidic fraction induced a strong increase in the number of IgA⁺ B cells in the intestinal lamina propria and total intestinal IgA secretion, as well as a significant increase in serum IgA. Interestingly, although the WPI and the two other peptide fractions (F1 and F2) had an impact in non-infected groups, they showed no effect on IgA secretion when mice were infected, since no significant difference was observed compared with the PBS group. This may be due to the fact that only total systemic IgA antibodies were assessed here, and the effects of the WP1, F1 and F2 might have been visible if anti-E. coli O157:H7-specific IgA had been measured. It could be hypothesized that the basic peptides exerted their effect through a different pathway: for instance, they could act in the intestine lumen by inhibiting bacterial adhesion or toxin transport, they might have a different receptor either on intestinal epithelial cells or on lymphocytes. Several hypothesis have been proposed for the mechanism of action of antimicrobial peptides: they can disrupt the cytoplasmic membrane of microorganisms, resulting in pore formation, and some have been shown to translocate across the membrane and affect cytoplasmic reactions. Host defence peptides also have the ability to modulate inflammation as well as the innate and adaptive immune responses. This could account for the systemic effects observed in the present study with the impacts on serum IgA and cytokine secretion.

Effect of the whey protein isolate and peptide fractions on serum cytokine production. Serum cytokines in infected groups were measured on days 1 and 7 (Table 6). When the mice were challenged with E. coli, IFN-γ levels rose after 24 hours then dropped significantly (p<0.01) on day 7 in the PBS control and WPI groups (data not shown). The same tendency of IFN-γ levels decrease after 7 day post-infection was observed for the peptide fractions-treated groups that did not differ statistically from the PBS group, even F2 that showed an effect in the non-infected mice. Even though the WPI had no impact in the non-infected mice, it significantly lessened the decrease in serum IFN-γ 7 days post-infection. This could be attributed to its glycomacropeptide (GMP) content (16.7%). Nakajima et al. (2005) suggested that GMP could play a role in the prevention of intestinal infection. They showed that GMP could bind EHEC O157 in vitro, and that this ability was linked to the carbohydrate chains. It also inhibited EHEC O157 adhesion to Caco-2 cells in a dose-dependent manner. Another possibility could be the immunoglobulin content of the WPI. An immunoglobulin enriched bovine colostrum provided protection against EHEC O157:H7 in mice pretreated with streptomycin, and prevented the attachment of EHEC O157:H7 to the sections prepared from the mice cecum walls. Lactoferrin was also showed to influence IFN-γ production. In a herpes simplex virus type 1 infection murine model, IFN-γ levels were higher in mice fed lactoferrin (vs water) 5 days after infection. Our results have shown that in non-infected mice, F1 inhibits IFN-γ production whereas F2 (and to a lesser degree, F3) stimulates it. Their effects are more visible on day 7 than immediately after the end of the gavage period. In infected mice, the WPI attenuates the drop in IFN-γ observed on day 7. This could be important in the context of the infection since IFN-γ functions include the activation of macrophages and of the expression of class II MHC on antigen presenting cells. IFN-γ stimulated macrophages are more phagocytic, they are more capable of killing intracellular pathogens and they have increased ability to present antigen. An increase in IFN-γ could result in heightened immune surveillance and immune system function during infection, when IFN-γ participates in an amplification loop to increase immune system sensitivity and response to pathogens. IFN-γ has the ability to activate macrophages and to regulate the expression of MHC class I and II proteins, hence it plays a role in both innate and adaptive immune responses.

Serum TGF-β1 was also measured in infected and non-infected groups (Table 6). However in infected mice, unfortunately we were limited by the number of remaining serum aliquots, so we did not have enough repetitions to see a significant difference between the treated and control groups. However our results in non-infected mice show that the basic peptide fraction F3 can stimulate TGF-β1 and that this effect is maintained until day 7. TGF-β1's roles are not limited to tolerance induction, it may also play a role during infection. It not only down-regulates immune responses and B cell proliferation, it also induces apoptosis of immature or resting B cells, and blocks B cell activation and class switching to most isotypes except for IgA. Thus the stimulation of IgA production by F3 is reflected in the increased TGF-β1 induced by this peptide fraction, and the inhibition of TGF-β1 by the WPI in non-infected mice on day 7 might be related to the slight decrease in serum IgA.

The present study showed that oral administration of a whey protein isolate and whey peptide fractions derived from its trypsin-chymotrypsin hydrolysate have the ability to modulate components of the immune response both in a non-infected and an E. coli-infected murine model. All whey protein and peptide products induced an increase in systemic total IgAs in the absence of an infection, suggesting a potential use as adjuvants. The measure of serum cytokine levels reflected our previous in vitro findings, especially with the enhanced IFN-γ production in the F2-treated groups. The basic F3 peptide fraction showed particularly promising results: it stimulated serum TGF-β1 secretion, which was correlated with a significant increase in IgA levels.

### Example III

### Identification of Immunomodulating Peptides Resulting from the Enzymatic Hydrolysis of Whey Proteins

Our previous work showed that a whey protein isolate (WPI), its enzymatic digest and peptide fractions separated from the hydrolysate had an impact on murine splenocyte proliferation in vitro. In addition, the peptide fractions oriented the cytokine secretion towards a Th1 profile, especially the peptide fractions with acidic or neutral pI. The immunomodulating properties of the WPI and the peptide fractions were further investigated in vivo: the whey products were given orally to mice in a healthy and infected model, and serum immunoglobulin and cytokine levels were assessed. In healthy mice, all whey products strongly stimulated serum IgA production, which could indicate a state of heightened immunological vigilance, whereas in the infected mice only the basic peptide fraction seemed to influence the measured immune parameters. The objective of the present study is to identify the whey peptides responsible for the observed immunomodulating effects.

Whey peptide fractions preparation and peptide source. Commercial microfiltered whey protein isolate (WPI) was kindly provided by Immunotech (Vaudeuil-Dorion, QC, Canada). The peptide fractions were prepared as previously described above. Briefly, the WPI was hydrolyzed using a mixture of trypsin:chymotrypsin (1:1), and the reaction was stopped by ultrafiltration when the degree of hydrolysis reached 10%. The resulting hydrolysate was subsequently fractionated by liquid-phase isoelectric focusing a preparative Rotofor cell (Bio-Rad Laboratories, Hercules, Calif., USA). When voltage and current were stabilized, twenty peptide fractions were collected and their pH was measured immediately (e.g., before dropping due to atmospheric CO₂ absorption), then the fractions were pooled into three peptide fractions according to their pH: F1 (pH<4.5), F2 (4.5<pH<7), and F3 (pH>7).

All β-lactoglobulin peptides tested on the murine splenocyte cultures were custom synthesized by AnaSpec, Inc. (San Jose, Calif.) and were reported to have >90% purity.

Whey peptide fractions characterization. Peptides in the hydrolysate and the 3 fractions were identified by liquid chromatography-mass spectrometry (LC-MS). RP-HPLC analyses were performed using a LC-MSD QUAD Agilent 1100 Series (Agilent Technologies, Palo Alto, Calif., USA) consisting of an autosampler (G1329A), two pumps (bin G1323A) and a diode array detector (DAD G1315A) adjusted to 214 nm. Peptides were analyzed with a Luna 5 μm C₁₈(2) column (2 i.d.×250 mm, Phenomenex, Torrance, Calif., USA) using the following conditions: flow rate, 0.2 mL min⁻¹; column temperature, 40° C.; solvent A, trifluoroacetic acid (TFA) 0.11 vol. % in water; solvent B, acetonitrile:water:TFA 90:/10:0.1 vol. %. Elution was obtained with a linear gradient of solvent B from 1 to 100 vol. % over 140 min. To reduce the effect of TFA, mass spectrometry was performed after infusing (50 μL min⁻¹) a mixture of 12% propionic acid and 12% isopropanol to the existing flow before the MS interface. Signals were recorded in positive mode using a 90 V fragmentation with a scan range of 200-3000 m/z. Nitrogen was used as the drying gas at 13 L min⁻¹ and 350° C., and as nebulizer gas at 0.241 MPa. The capillary voltage was set at 3000 V. The instrument was calibrated using an ES tuning mix (G2431A, Agilent). The detected molecular weights were matched with the expected molecular weights of peptides resulting from tryptic-chymotryptic digestion according to the Compute pI/Mw Tool available from the ExPASy proteomics server.

In vitro murine splenocyte proliferation assay. The impact of whey peptides on splenocyte proliferation was assessed as follow. Spleens from 6-week-old female BALB/c mice (Charles River, St-Constant, QC, Canada) were removed, mechanically disrupted in complete RPMI-1640 medium (RPMI-1640 medium supplemented with 2 mmol L⁻¹ L-glutamine (Wisent, St-Bruno, QC, Canada), 10% foetal calf serum (FCS, Wisent), 50 μmol L⁻¹ mercaptoethanol (Sigma-Aldrich), and 100 μg mL⁻¹ of penicillin/streptomycin (Wisent), and then filtered through 70 μm cell strainer (BD Falcon, Bedford, Mass., USA) under aseptic conditions. Erythrocytes were lysed by osmotic shock using 0.87% NH₄Cl (Sigma-Aldrich) for 2 min at 37° C., then cells were washed 3 times at 4° C. with complete RPMI. They were stained with 0.4% trypan blue (Celigro, Mediatech, Washington, D.C., USA) and counted using a Malassez chamber. The cell suspension was then adjusted to 1.25×10⁶ viable cells mL⁻¹ in RPMI medium.

The cells (100 μL) were added to the wells of 96-well round-bottomed microplates containing 50 μL of RPMI, or 50 μL of peptide solutions. Various concentrations (10-2000 μg mL⁻¹) of the solutions, which were prepared in complete RPMI and adjusted to pH 7, were added to the wells. The same experiments were also performed in the presence of ConA (0.5 μg mL⁻¹) to evaluate the effects of the products on ConA-stimulated cells. The microplates with and without ConA were incubated for 72 h and 96 h, respectively. Cell proliferation was evaluated by the reduction of the fluorochrome Alamar Blue™ (BioSource international, Camarillo, Calif., USA), which was added to the wells (20 μL) for the last 24 h of the incubation periods. The fluorescence of the supernatants was monitored at an excitation wavelength of 544 nm and an emission wavelength of 590 mm using a fluorometer (Fluoroscan Ascent, Thermo Electron Inc., Milford, Mass., USA). Data are expressed as a stimulation index (SI).

Cytokine analyses. After 72 hours, the supernatants from the splenocyte cultures with the cells, cells+ConA (0.5 μg/mL), and each of the peptides (2000 μg/mL) in the presence of ConA (0.5 μg/mL) were collected and stored at −20° C. until used for the cytokine detection assay. A cytokine profile analysis was performed by using the RayBio Mouse Cytokine Antibody Array G series 2 (RayBiotech, Inc., Norcross, Ga.), in order to obtain semi-quantitative levels of 32 different growth factors, cytokines and chemokines (Table 7).

TABLE 7
List of cytokines detected on the mouse cytokine membrane array.

| Pos 1 | Pos 2 | Pos 3 | Neg | Neg | 6Ckine | CTACK | Eotaxin | GCSF | GM-CSF |
|---|---|---|---|---|---|---|---|---|---|
| IL-2 | IL-3 | IL-4 | IL-5 | IL-6 | IL-9 | IL-10 | IL-12 | IL-12 | IL-13 |
| | | | | | | | p40p70 | p70 | |
| IL-2 | IL-3 | IL-4 | IL-5 | IL-6 | IL-9 | IL-10 | IL-12 | IL-12 | IL-13 |
| | | | | | | | p40p70 | p70 | |
| IL-17 | IFN-γ | KC | Leptin | MCP-1 | MCP-5 | MIP-1α | MIP-2 | MIP-3 β | RANTES |
| IL-17 | IFN-γ | KC | Leptin | MCP-1 | MCP-5 | MIP-1α | MIP-2 | MIP-3 β | RANTES |
| SCF | sTNFRI | TARC | TIMP-1 | TNF-α | Thrombo | VEGF | Neg | Neg | Neg |
| | | | | | poietin | | | | |
| SCF | sTNFRI | TARC | TIMP-1 | TNF-α | Thrombo | VEGF | Neg | Neg | Neg |
| | | | | | poietin | | | | |

Incubation protocol, signal detection, and interpretation of the data were performed according to manufacturer instructions. Results were expressed as the relative quantity of cytokines detected in the culture supernatants calculated according to the following equation:

$$Cytokine\_Index = \frac{{Intensity}_{sample} - {Background}_{Sample}}{{Intensity}_{cells + ConA} - {Background}_{Cells + ConA}} \qquad [ 3 ]$$

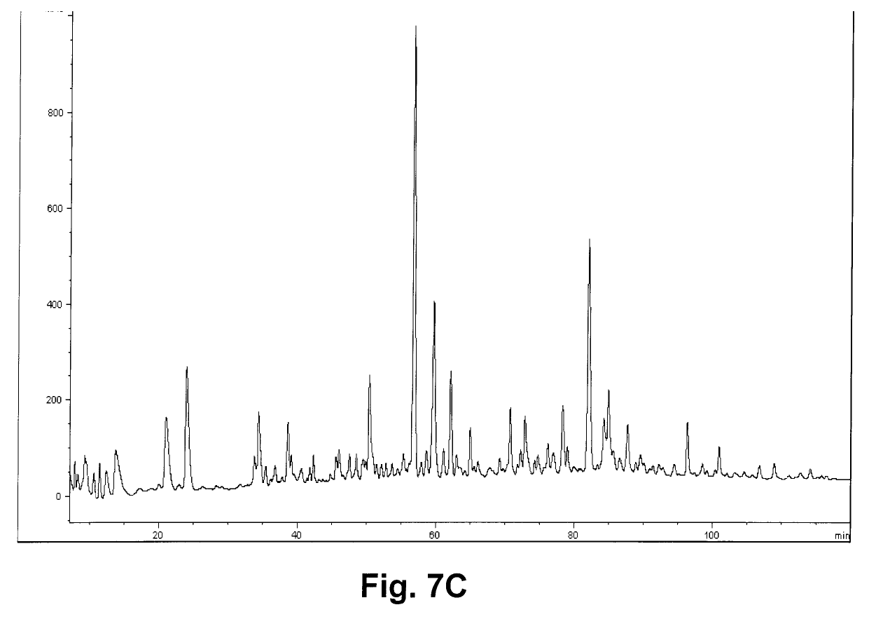

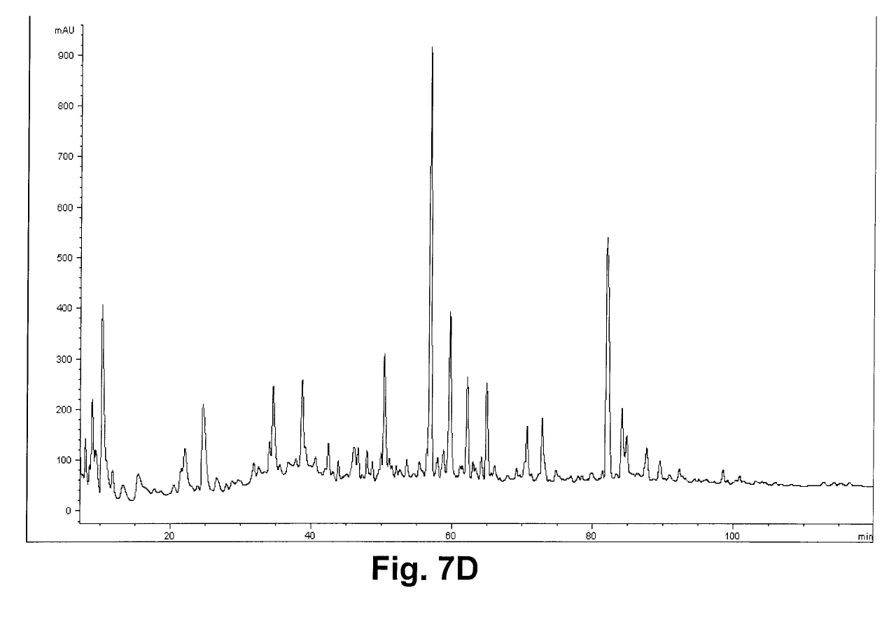

Characterization of the hydrolysate and peptide fractions by LC-MS. The hydrolysate and the three peptide fractions were analyzed by LC-MS for peptide identification and their RP-HPLC chromatograms are illustrated in FIG. 7. Peak numbers on this figure refer to identified peptides obtained by LC-MS as described in Table 8. The hydrolysate results from the trypsin and chymotrypsin hydrolysis of a whey protein isolate containing 46.9% β-lactoglobulin, 26.8% α-lactalbumin and 16.7% GMP. Therefore it is likely that most of the peptides present in the hydrolysate correspond to fragments of these proteins, and not the minor proteins of the isolate. Out of the 45 peaks in the hydrolysate chromatogram (FIG. 7A), 26 corresponded to β-lactoglobulin fragments, 10 were α-lactalbumin fragments, and 9 remained unidentified.

The hydrolysate was separated into three fractions by isoelectric focusing: pI<4.5 for F1, 4.5<pI<7 for F2 and pI>7 for F3. This fractionation according to their isoelectric point (pI) is somewhat apparent in the chromatograms. For example peptides with an acidic pI such as β-Lg f41-42, f125-135, f125-133, f42-60, and f21-32 were only found in the hydrolysate and F1. Similarly, peptides with a more basic pI such as β-Lg f139-141 and f146-148 were only present in the hydrolysate and F3. However some peptides have ended up in a fraction that does not correspond to their own pI, or appear in all fractions such as α-La (f51-53) (peak # 30), β-Lg f78-82 (peak # 33) and β-Lg f103-105 (peak # 36). Even though these three peptides all have a very low pI and therefore should have been found only in the F1 fraction, the isoelectric focusing failed to separate them, either because of their lack of charge at pH 7 or because of peptide-peptide interactions. Groleau et al. (2002) also observed a poor separation of two uncharged peptides both individually and in mixture with other peptides. Their work showed no peptide-peptide interactions in the IEF separation, and the authors concluded that the absence of charge was the main factor.

TABLE 8
Identification by LC-MS of the peptide peaks present (+) in the whey protein
hydrolysate, the fractions F1 (pI < 4.5), F2 (4.5 < pI < 7) and F3 (pI > 7).

| Pea | Peptidi | Amino Acid | Experi | Theor | pI^{a} | Net | HΦₐᵥₑ | Hy | F | F | F |
|---|---|---|---|---|---|---|---|---|---|---|---|
| 1 | β-Lg | (K)ALK | 330.2 | 330.4 | 8.8 | +1 | 1.55 | | | | + |
| 2 | β-Lg | (M)HIR | 424.3 | 424.5 | 9.7 | +1 | 1.23 | | | | + |
| 3 | β-Lg | (K)FDK | 408.2 | 408.4 | 5.8 | 0 | 1.38 | | | + | + |
| 4 | α-La | (L)CEK | 378.2 | 378.4 | 5.9 | 0 | 0.83 | + | | | |
| 5 | β-Lg | (L)ACQCL | 535.0 | 536.7 | 5.5 | 0 | 1.03 | + | | | |
| 6 | α-La | (F)QINNK | 615.3 | 615.7 | 8.7 | +1 | 0.89 | + | | | |
| 7 | α-La | EQL | 387.3 | 388.4 | 4.0 | −1 | 0.8 | + | | | |
| 8 | β-Lg | (K)ALK | 330.2 | 330.4 | 8.8 | +1 | 1.55 | + | | | |
| 9 | α-La | (F)HTSGY | 563.3 | 563.6 | 6.7 | 0 | 0.66 | + | | + | + |
| 10 | β-Lg | (M)HIR | 424.2 | 424.3 | 9.7 | +1 | 1.23 | + | | + | + |
| 11 | β-Lg | (K)FDK | 408.2 | 408.4 | 5.8 | 0 | 1.38 | + | | | |
| 12 | α-La | (F)HTSGY | 563.2 | 563.6 | 6.7 | 0 | 0.66 | + | | | |
| 13 | β-Lg | (R)VY | 280.1 | 280.3 | 5.4 | 0 | 2.28 | + | + | | |
| 14 | β-Lg | (K)IIAEK | 572.4 | 572.7 | 6.0 | 0 | 1.63 | + | | + | + |
| 15 | β-Lg | (L)DAQSAPLR | 856.5 | 856.9 | 5.8 | 0 | 0.91 | + | | + | + |
| 16 | α-La | (L)DDDL | 476.8 | 476.4 | 3.4 | −3 | 0.60 | + | | + | + |
| 17 | β-Lg | (K)IDALNENK | 915.7 | 916.0 | 4.3 | −1 | 0.95 | + | + | + | + |
| 18 | β-Lg | (K)KIIAEK | 700.4 | 700.9 | 8.5 | +1 | 1.61 | + | | + | + |
| 19 | | | 852.5 | | | | | + | + | | |
| 20 | β-Lg | (K)GLDIQK | 672.4 | 672.8 | 5.8 | 0 | 1.14 | + | | + | + |
| 21 | β-Lg | (R)TPEVDDEALEK | 1245.0 | 1245. | 3.8 | −4 | 0.85 | + | + | | |
| 22 | β-Lg | (K)VAGTW | 532.3 | 532.6 | 5.4 | 0 | 1.18 | + | | + | + |
| 23 | α-La | (K)DL | 246.1 | 246.3 | 3.8 | −1 | 1.20 | + | | + | + |
| 24 | α-La | (K)LDQW | 560.2 | 560.6 | 3.8 | −1 | 1.35 | + | + | + | |
| 25 | α-La | (K)IW | 317.2 | 317.4 | 5.5 | 0 | 2.98 | + | | + | + |
| 26 | β-Lg | (L)DTDY | 512.3 | 512.5 | 3.5 | −2 | 0.83 | + | | + | + |
| 27 | | | 474.3 | | | | | + | + | | |
| 28 | β-Lg | (K)VLVLDTDYKK | 1191.5 | 1193. | 5.9 | 0 | 1.45 | + | + | | |
| 29 | β-Lg | (K)VAGTWY | 695.3 | 695.8 | 5.4 | 0 | 1.46 | + | | + | + |
| 30 | α-La | (Y)GLF | 335.2 | 335.4 | 5.5 | 0 | 1.68 | + | + | + | + |
| 31 | | | 2665.5 | | | | | + | + | + | |
| 32 | | | 1900.5 | | | | | + | + | | |
| 33 | β-Lg | (K)IPAVF | 545.3 | 545.7 | 5.5 | 0 | 2.13 | + | + | + | + |
| 34 | β-Lg | (L)DAQSAPL | 700.5 | 700.75 | 3.80 | −1 | 0.93 | + | + | + | + |
| 35 | f33-39 | | 700.5 | | | | | | + | | |
| 36 | β-Lg | (Y)LLF | 391.3 | 391.5 | 5.5 | 0 | 2.48 | + | + | + | + |
| 37 | β-Lg | (L)DAQSAPL | 700.6 | 700.7 | 3.8 | −1 | 0.93 | | | + | + |
| 38 | β-Lg | (R)TPEVDDEAL | 990.5 | 988.0 | 3.4 | −4 | 0.88 | + | + | | |
| 39 | | | 1681.6 | | | | | + | + | | |
| 40 | | | 998.6 | | | | | + | + | | |
| 41 | | | 3088.1 | | | | | + | | + | |
| 42 | β-Lg | (Y)VEELKPTPEGDLE | 2051.0 | 2051. | 4.2 | −3 | 1.27 | + | + | | |
| 43 | β-Lg | (Y)SLAMAASDISLL | 1190.6 | 1191. | 3.8 | −1 | 1.14 | + | + | | |
| 44 | | | 1297.3 | | | | | + | + | | |
| 45 | | | 1422.6 | | | | | + | + | + | |

Peak numbers refer to FIG. 1.
^{a}Isoelectric points were calculated using the ExPASy Molecular Biology Server.
^{b}Amino acid preceding the scissile bond is shown in parenthesis ( ).
^{c}Average hydrophobicity was calculated according to the method of Bigelow (1967).

We have previously reported that F3 was the only peptide fraction able to stimulate the production of both serum TGF-β1 and IgA in a murine model of infection by E. coli O157:H7. In order to determine which peptide or peptides unique to the basic fraction could potentially be responsible for this effect, the chromatograms of the three peptide fractions were compared. Four peptides present only in F3 (or mainly in F3 and as minor components in F2) were identified and synthesized: β-Lg f139-141 (peak # 1), β-Lg f146-148 (peak # 2), (f9-14) (peak # 20) and β-Lg f96-99 (peak # 26).

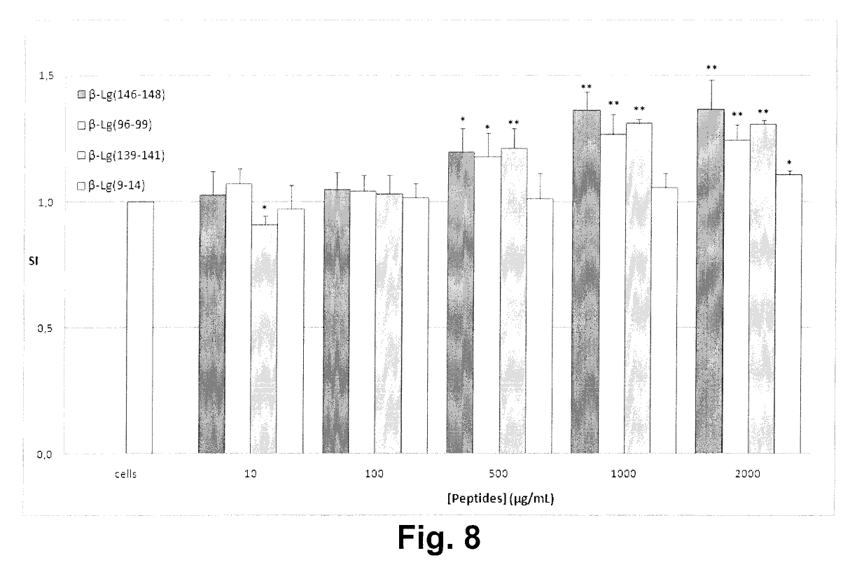

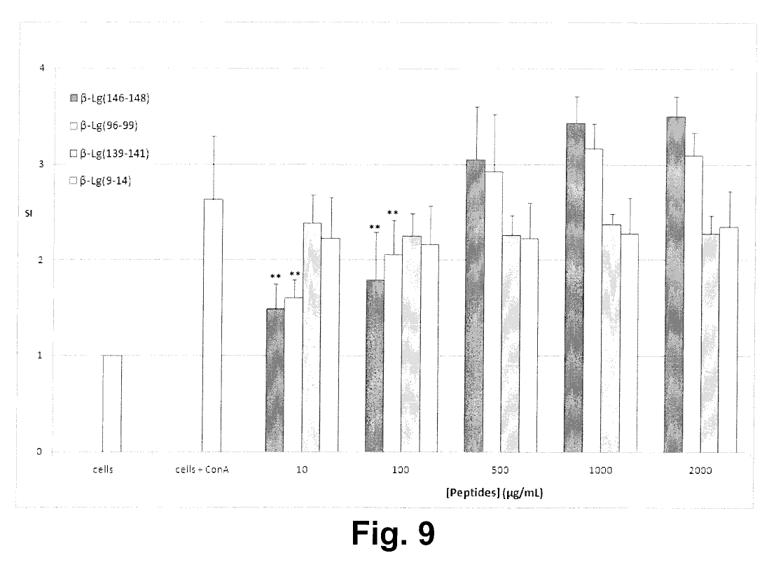

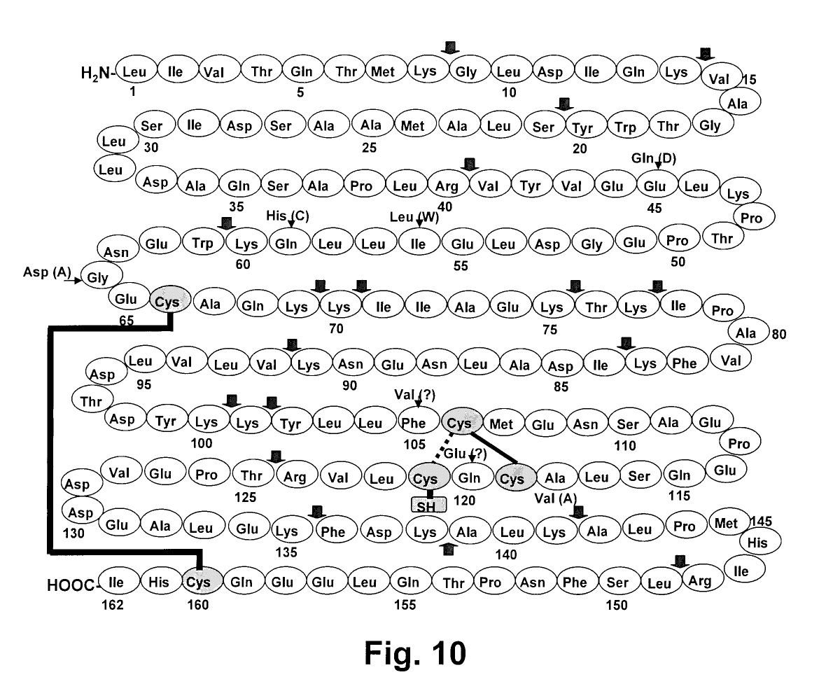

Impact of F3 peptides on splenocyte proliferation. The effect of the four selected peptides on murine splenocyte proliferation was evaluated in the absence (FIG. 8) and presence of ConA (FIG. 9). The β-Lg f146-148, β-Lg f96-99 and β-Lg f139-141 all stimulated the proliferation of resting cells (FIG. 9) in a dose-dependent manner up to the highest concentration used (2000 μg/mL), this effect becoming significant at 500 μg/mL. The Si values were more important for the β-Lg f146-148 than for the other peptides, suggesting a higher immunostimulating capacity. They were equivalent or slightly higher than the SI values obtained with the whole F3 fraction at 500 μg/mL (1.20 vs. 1.12 for F3), 1000 μg/mL (1.36 vs. 1.20 for F3) and 2000 μg/mL (1.37 vs. 1.39 for F3). The β-Lg f9-14 showed only a small stimulation at the highest concentration (2000 μg/mL).

In the presence of ConA, the β-Lg f146-148 (SEQ ID NO. 4), β-Lg f96-99 (SEQ ID NO. 6), β-Lg f139-141 (SEQ ID NO. 2) and β-Lg f9-14 (SEQ ID NO. 8) all lost their stimulating capacity at higher concentrations. The SI tended to increase with β-Lg f146-148 (SEQ ID NO. 4), but this effect did not reach significance due to larger variations between repetitions. This is similar to what was observed with the F3 fraction, which required higher concentrations (2000 and 4000 μg/mL) to stimulate significantly splenocyte proliferation when ConA was added to the culture medium. Although the β-Lg f146-148 (SEQ ID NO. 4) and β-Lg f96-99 (SEQ ID NO. 6) had no effect at low concentrations in resting splenocytes, they inhibited ConA-induced proliferation at 10 and 100 μg/mL.

Very few studies have assessed the impact of individual whey peptides on lymphocyte proliferation. Two synthetic peptides corresponding to the sequences α-La f50-51 (Tyr-Gly) and α-La f18-20 (Tyr-Gly-Gly) increased the in vitro proliferation of ConA-stimulated human peripheral blood lymphocytes (Kayser & Meisel, 1996). We previously assessed the effect of six β-Lg peptides and one α-La peptide (chosen based on their amino acid composition, molecular weight, hydrophobicity and charge) on murine splenocyte cultures. All of them were found to stimulate the proliferation of resting cells. When the splenocytes were stimulated by ConA, the same tendencies were observed although, like in the present study, higher concentrations were required to see the effects. Three peptides assessed in example I overlap or include some of the peptides tested in the present study, which may help pinpoint the active sequences within the original β-Lg protein. They observed a stimulation of splenocyte proliferation by β-Lg f92-105 (SEQ ID NO. 5), and our results suggest that this effect could be attributed to the β-Lg f96-99 (SEQ ID NO. 6) fragment. Similarly, the fragments β-Lg f139-141 (SEQ ID NO. 2) and β-Lg f146-148 (SEQ ID NO. 4) may account for the stimulatory capacity of β-Lg f139-148 (SEQ ID NO. 1) and β-Lg f142-148 (SEQ ID NO. 3), respectively. The peptides β-Lg f142-148 (SEQ ID NO. 3), β-Lg f142-145, β-Lg f146-148 and β-Lg f147-148 have also been identified as ACE-inhibitory and antihypertensive, suggesting multiple bioactivities located in the same region of the β-lactoglobulin.

Several whey protein components have been shown to have inhibitory effects. Lactoferrin and lactoperoxydase inhibit the proliferation of sheep blood lymphocytes in response to ConA, but this effect was decreased or suppressed for the whey protein concentrate or when the two proteins were combined. GMP inhibits the mitogen-induced proliferative responses of murine splenocytes. Cross & Gill (1999) showed an inhibition of mitogen-stimulated lymphocyte proliferation by a whey protein concentrate rich in GMP, but the effect was lost after digestion by pepsin and pancreatin.

Impact of F3 peptides on cytokine secretion. Of the 32 different cytokines, chemokines and growth factors, which were measured in the splenocyte supernatants after incubation with each of the four peptides, seven were expressed substantially (Table 9).

TABLE 9
Relative quantity of cytokines detected in the supernatants
for each of the β-Lg peptides tested on murine splenocytes
cultured in the presence of ConA (0.5 μg/mL).

| | | β-Lg f146- | β-Lg f96- | β-Lg f139- | β-Lg f33- | β-Lg f9- |
|---|---|---|---|---|---|---|
| | Cells + | 148 + | 99 + | 141 + | 40 + | 14 + |
| | ConA | ConA | ConA | ConA | ConA | ConA |
| IL-2 | 1 | 2.1 | 3.6 | 1.7 | 0.6 | 2.6 |
| IL-3 | 1 | 1.8 | 2.8 | 0.7 | 0.1 | 1.0 |
| IL-4 | 1 | 0.5 | 1.8 | 1 | 0.7 | 1.4 |
| IL-17 | 1 | 1.4 | 1.7 | 1.1 | 0.6 | 1.1 |
| IFN-γ | 1 | 8.6 | 4.5 | 1.0 | 0.7 | 1.7 |
| RANTES | 1 | 0.8 | 1.4 | 1.7 | 0.4 | 1.0 |
| VEGF | 1 | 4.8 | 2.3 | 0.9 | 0.8 | 2.0 |

IL-2 expression was enhanced by almost all peptides, which could account for their effect on splenocyte proliferation. On the other hand, several of the cytokines measured for β-Lg f139-141 (SEQ ID NO. 2) had a cytokine index close to 1, suggesting a cytokine profile similar to the control. β-Lg f146-148 (SEQ ID NO. 4) strongly inhibited IL-4 and induced a very high expression of IFN-γ. Out of the four peptides tested, β-Lg f146-148 (SEQ ID NO. 4) exhibited effects similar to the F3 peptide fraction, which also significantly increased IFN-γ by murine splenocytes in the presence of ConA. IL-3 and IL-4 levels were increased by β-Lg f96-99 (SEQ ID NO. 6) and β-Lg f9-14 (SEQ ID NO. 8), β-Lg f139-141 (SEQ ID NO. 2) also stimulated the production of IL-17, which could indicate an orientation towards a Th17 phenotype. TGF-β is required for the differentiation of naive T cells into inflammatory Th17 cells. TGF-β was not measured here, but it has been demonstrated that the oral administration of a peptide fraction containing all four peptides significantly stimulated serum TGF-β1 production in mice. This may explain the slightly enhanced IL-17 secretion observed in the present study. The chemotactic cytokine RANTES (CCL5) was slightly increased in the presence of β-Lg f96-99 (SEQ ID NO. 6) and β-Lg f139-141 (SEQ ID NO. 2). Vascular endothelial growth factor (VEGF) was increased by β-Lg f9-14 (SEQ ID NO. 8) and β-Lg f96-99 (SEQ ID NO. 6), and strongly stimulated by β-Lg f146-148 (SEQ ID NO. 4). VEGF plays a role in developmental, physiological, and pathological vasculogenesis and angiogenesis. It can induce angiogenesis in the absence of inflammation, but its expression has also been associated with inflammatory diseases such as atherosclerosis and arthritis. VEGF is expressed in inflammatory peritoneal macrophages, which also produced high levels of IFN-γ. Moreover, VEGF's proinflammatory action is mediated by IFN-γ, since it induces expression and function of the T cell chemoattractant chemokine IFN-γ-inducible protein of 10 kDa (IP-10). In human monocytes and macrophages, IFN-γ increased VEGF expression. Therefore the high expression of VEGF in the presence of β-Lg f146-148 (SEQ ID NO. 4) might be related to the important production of IFN-γ induced with this peptide.

Taken as a whole, the results suggest three distinct profiles for the peptides evaluated: β-Lg f9-14 (SEQ ID NO. 8) and β-Lg f96-99 (SEQ ID NO. 6) stimulated the production of IL-3 and IL-4 and oriented the splenocytes towards a Th2 response, β-Lg f139-141 (SEQ ID NO. 2) seemed neutral, and β-Lg f146-148 (SEQ ID NO. 4) induced a Th1 profile with a strong IFN-γ production and an inhibition of IL-4.

Whey proteins are known precursors of bioactive peptides liberated by digestive enzymes. These results demonstrate that five short-chained peptides derived from bovine β-lactoglobulin are able to modulate the proliferation of resting and mitogen-stimulated murine splenocytes. Their mechanism of action was further characterized by their effect on cytokine secretion in the same in vitro model. All four peptides can potentially be released during the trypsin-chymotrypsin enzymatic digestion of the β-lactoglobulin, but they exert extremely different impacts on murine splenocytes ranging from neutral for β-Lg f139-141 (SEQ ID NO. 2) to a strong stimulation of the proliferation with an orientation towards a Th1 cytokine profile for β-Lg f146-148 (SEQ ID NO. 4) or a Th2 profile for β-Lg f9-14 (SEQ ID NO. 8) and β-Lg f96-99 (SEQ ID NO. 6). Such a variety of effects opens numerous possibilities for potential applications, either as nutraceuticals or included in functional foods. Since the sequence HIR (β-Lg f146-148; SEQ ID NO. 4) appears to stimulate Th1 cytokines, it might prove useful in the prevention of some infections or in the management of allergy. Alternatively, since the sequences GLDIQK (β-Lg f9-14; SEQ ID NO. 8) and DTDY (β-Lg f96-99; SEQ ID NO. 6) seem to favour a Th2 cytokine profile, they may be used in the context of inflammatory disease. Additional research is needed to further characterize their mechanism of action as well as evaluate their stability during the gastrointestinal passage and their level of absorption in order to determine a safe and efficient dose and eventually confirm these effects in vivo.

While the invention has been described with particular reference to the illustrated embodiment, it will be understood that numerous modifications thereto will appear to those skilled in the art. Accordingly, the above description and accompanying drawings should be taken as illustrative of the invention and not in a limiting sense.

1-36. (canceled)

37. A composition comprising a peptide selected from the group consisting of: SEQ ID NO:2 optionally comprising up to seven additional amino acids at the carboxy-terminal end; SEQ ID NO:4 optionally comprising up to seven additional amino acids at the amino-terminal end; SEQ ID NO:3 optionally comprising up to three additional amino acids at the amino-terminal end; SEQ ID NO:1; SEQ ID NO:6 optionally comprising up to four additional amino acids at the amino-terminal end and up to six additional amino acids at the carboxy-terminal end; SEQ ID NO:7 optionally comprising up to four additional amino acids at the amino-terminal end and up to five additional amino acids at the carboxy-terminal end; SEQ ID NO:5; SEQ ID NO:9 optionally comprising up to two additional amino acid at the amino-terminal end; and SEQ ID NO:8; or a salt thereof.

38. An adjuvant comprising the composition of claim 37.

39. A composition according to claim 37, selected from the group consisting of: SEQ ID NO:2 optionally comprising up to seven additional amino acids at the carboxy-terminal end; SEQ ID NO:4 optionally comprising up to seven additional amino acids at the amino-terminal end; SEQ ID NO:3 optionally comprising up to three additional amino acids at the amino-terminal end; and SEQ ID NO:1; or a salt thereof, wherein said composition increases the secretion of a Th1-cytokine by immune cells.

40. The composition of claim 39, wherein the Th1-cytokine is selected from the group consisting of: IL-2, INF-γ, TNF-α and GM-CSF.

41. A method for inducing a Th1 response in a subject in need thereof, said method comprising the step of administering an immunostimulatory-effective amount of the composition of claim 39 to the subject.

42. A method for treating or preventing an infection in a subject in need thereof, said method comprising the step of administering an immunostimulatory-effective amount of the composition of claim 39 to the subject.

43. The method of claim 42, wherein the infection is caused by a microorganism selected from the group consisting of Escherichia coli, Streptococcus pneumoniea, Clostridium difficile and influenza virus.

44. A method for treating or preventing an allergy in a subject in need thereof, said method comprising the step of administering an immunostimulatory-effective amount of the composition of claim 39 to the subject.

45. The method of claim 44, wherein the allergy is selected from the group comprising food allergy, pollen allergy, dust mites allergy and allergy to animal-derived allergens.

46. The method of claim 41, wherein the administration is selected from the group consisting of: oral administration, nasal administration, enteral administration, rectal administration, vaginal administration and transmucosal administration.

47. The method of claim 46, wherein the subject is human.

48. A composition selected from the group consisting of: SEQ ID NO:6 optionally comprising up to four additional amino acids at the amino-terminal end and up to six additional amino acids at the carboxy-terminal end; SEQ ID NO:7 optionally comprising up to four additional amino acids at the amino-terminal end and up to five additional amino acids at the carboxy-terminal end; SEQ ID NO:5; SEQ ID NO:9 optionally comprising up to two additional amino acid at the amino-terminal end; and SEQ ID NO:8, or a salt thereof, wherein said composition increases the secretion of a Th2-cytokine by immune cells.

49. The composition of claim 48, wherein the Th2-cytokine is selected from the group consisting of: IL-4, IL-5, IL-6, IL-10, IL-13 and TGF-β.

50. A method for inducing a Th2 response in a subject in need thereof, said method comprising the step of administering an immunostimulatory-effective amount of the composition of claim 48 to the subject.

51. A method for stimulating the production of immunoglobulin A in a subject in need thereof, said method comprising the step of administering an immunostimulatory-effective amount of the composition of claim 48 to the subject.

52. A method for treating or preventing an inflammatory disease in a subject in need thereof, said method comprising the step of administering an immunostimulatory-effective amount of the composition of claim 48 to the subject.

53. The method of claim 51, wherein the inflammatory disease is selected from the groups consisting of: a VEGF-associated inflammatory disease, atherosclerosis, eczema, inflammatory bowel disease, and arthritis.

54. The method of claim 50, wherein the administration is selected from the group consisting of: oral administration, nasal administration, enteral administration, rectal administration, vaginal administration and transmucosal administration.

55. The method of claim 54, wherein the subject is human
